(19) United States
(12) Patent Application Publication
(10) Pub. No.: US 2025/0116035 A1
(43) Pub. Date: Apr. 10, 2025

(54) A METHOD FOR REPRODUCIBLE APTAMER SELECTION USING CLOSED SEQUENCE SOLUTION SPACES

(71) Applicant: NEOVENTURES BIOTECHNOLOGY INC., London (CA)

(72) Inventor: Gregory PENNER, Perwez (CA)

(73) Assignee: NEOVENTURES BIOTECHNOLOGY INC., London, ON (CA)

(21) Appl. No.: 18/730,366

(22) PCT Filed: Jan. 20, 2023
(86) PCT No.: PCT/CA2023/050070

§ 371 Date: Jul. 19, 2024

Related U.S. Application Data
(60) Provisional application No. 63/301,802, filed on Jan. 21, 2022.

(30) Foreign Application Priority Data
Jul. 28, 2022 (EP) 22187643.6

(51) Int. Cl. C40B 40/06; C12N 15/10; C12N 15/115

(52) U.S. Cl. CPC C40B 40/06; C12N 15/1048; C12N 15/115; C12N 2310/16; C12N 2320/12; C12N 2330/31

(57) ABSTRACT

In the field of aptamers, closed sequence solution space libraries for aptamer selection. Also, methods for selecting aptamers for binding to target molecules in which biological samples derived from individuals that differ by phenotype are contacted with the library of aptamers and the aptamer oligonucleotides that bound to the target molecules are covered. Further, methods of treating a disorder or a disease of a subject in which the disorder or disease is diagnosed using the library of aptamers and the subject is treated.

### FIELD OF THE INVENTION

The present invention relates to the field of aptamers, and provides a library of sequences for aptamer selection and related methods for selecting aptamers for binding to target molecules.

### BACKGROUND

Aptamer development has grown into a global industry since their invention over thirty years ago. A key difficulty with aptamer selection has been that it is essentially not reproducible as the sampling of sequences used for any given project represents a small sub-sample of all the possible sequences in a synthesized library. Also, the use of libraries with lower numbers of random nucleotides has been constrained by the need for primer recognition sites to support reiterative selection. The following invention provides a basis for the effective use of closed solution spaces of sequences for aptamer selection, first with relatively few random nucleotides for small molecules selection and secondly with sufficient random nucleotides to support sufficiently complex structures for larger molecules. Aptamers derived from this new process have been termed “Neomers” by the Inventor, which means than a library of aptamers can be defined as “Neomer Library.”

Aptamers are synthetic, single stranded oligonucleotides that mimic antibodies in their ability to act as ligands and bind to analytes. U.S. Pat. No. 5,475,096 A teaches a method for the in vitro selection of DNA or RNA molecules that are capable of binding specifically to any given target molecule. The method taught is composed of a process of reiterative selection steps that the inventors named “Systematic Evolution of Ligands by Exponential Enrichment” or SELEX.

The Inventors of U.S. Pat. No. 5,475,096 A suggested that only one cycle of selection may be sufficient in order to identify desirable aptamer sequences if the selection process was sufficiently stringent. Here, I quote from this patent,
“In one embodiment of the method of the SELEX Patent Applications, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.
In many cases, it is not necessarily desirable to perform the iterative steps of SELEX until a single nucleic acid ligand is identified. The target-specific nucleic acid ligand solution may include a family of nucleic acid structures or motifs that have a number of conserved sequences and a number of sequences which can be substituted or added without significantly effecting the affinity of the nucleic acid ligands to the target. By terminating the SELEX process prior to completion, it is possible to determine the sequence of a number of members of the nucleic acid ligand solution family.”

We define a closed solution space in terms of possible aptamer sequences as meaning an initial selection library that contains multiple copies of all possible sequences and the capacity to characterize the frequencies of all possible sequences.

In the US patent described above, this definition of closed solution space was not contemplated because this was prior to the invention and use of next generation sequencing. They only contemplated an open solution system because they did not consider it possible to characterize all of the possible sequences in the naïve selection library (the random library prior to the initiation of selection). Instead, they relied on either reiterative selection or highly effective selection to limit the number of successful aptamers (aptamers that bind to the target) to a smaller number than were present in the naïve library. There was no consideration of the possibility of comparing changes in frequency for every sequence in the naïve library to the frequency of every sequence in a selected library after a single round of selection. This necessary reduction in the number of sequences is a function of both the number of possible sequences in the naïve library, and the capacity to sequence the aptamers after selection. They clearly state that this problem can be solved with either extremely strong selection pressure or with reiterative selection. In this invention we provide an alternative solution which relies on the design of the selection library such that the frequency of all of the possible sequences in the naïve library can be characterized and thus can be directly compared to the frequency of all of the possible sequences in a selected library after a single round of selection, or after multiple rounds of selection.

The SELEX process disclosed in the original patent outlined in italics above requires synthesis with primer recognition sites that flank the random sequence on both the 5′ end and 3′ ends. These extended, conserved, sequence regions are required for this invention because they enable PCR amplification of the selected library. However, given that the sequences within the library are single-stranded, and given that a portion of these sequences are random, a large proportion of the sequences in the naïve library will exhibit substantial secondary structure complexation with random nucleotides and the primer recognition sites. This is deleterious for several reasons.

Reason 1: the selection process is based on the functionality of the aptamers (their ability to bind to a specific target). This functionality is manifested by the secondary and tertiary structure that the aptamers are able to adopt based on their sequences. We can think of this as the structure space for an aptamer library. The presence of extended primer recognition sequences constrains the secondary and tertiary structure space. The possible structure space is not fully random as it is dominated by structures that involve the primer recognition sequences.

In practice this difficulty is reduced by the employment of more random nucleotides, thus promoting the probability of a broader diversity of structures based on hybridization within the random region. This does not entirely overcome this deleterious effect however as still a large proportion of the sequences and consequently the structures do involve hybridization between nucleotides in the random region and the primer recognition region.

Reason 2: a broader diversity of structures can be obtained with the use of more random nucleotides but this also leads to longer aptamers, with the probability that sub-structures within an aptamer are responsible for its functionality and that other regions of the aptamer are not involved in binding. The presence of these functional regions in the selected aptamer is deleterious as these not only add to the cost of aptamer synthesis but they also have the potential to decrease aptamer functionality by interfering with the functional domain (decreasing affinity) or by exhibiting the capacity to bind to other target molecules (decreasing specificity). This deleterious effect affects aptamer selection for all targets but is of increasing concern as the size of the target molecule decreases. The smaller the target molecule the less nucleotides will be involved in binding events with the target, and thus the more profound effects of such non-necessary domains become in terms of affinity and specificity.

To explain further, it is common in the art of aptamer selection to use primer recognition regions that average 20 nucleotides in length flanking a random region of 40 nucleotides. As such, the selected full-length aptamers are 80 nucleotides in length. An aptamer of this length has a higher probability on average of being in flux between different possible shapes at the temperature at which it has been selected (FIGS. 1 & 2). An example of this flux in secondary structure of a random aptamer sequence is provided below as an example. This analysis is based on the use of software referenced by Gruber et al., 2008 (Nucleic Acids Res. 36 (Web Server issue): W70-W74).

This flux between possible shapes results in a decrease in the proportion of time that the aptamer is in a shape that is optimal for binding to the target molecule. This increases the probability that a proportion of the times that an aptamer collides with its target the aptamer will not be in the appropriate shape for binding to occur.

In the art, it is common to attempt to overcome this difficulty by truncating the aptamer to a minimal size and to limit the number of possible structures that can be formed. This practice is time-consuming, and not always successful, as simple truncation of a sequence often does not result in an improvement in the stability of a sub-structure. Such stabilization also requires the introduction of nucleotide substitutions, and given that these substitutions were not part of the original selection, they may result in complete loss of aptamer function.

Reason 3: we have disclosed an invention for an alternative approach to SELEX-based aptamer selection which we have named FRELEX (EP 3 344 805 B1 and U.S. Pat. No. 10,415,034 B2). In this approach, we expose traditional aptamer libraries to a field of 8 contiguous random nucleotide oligonucleotides immobilized on a surface. In phase I of selection, we select for aptamers with the capacity to hybridize with the random 8-mers, and discard all aptamers that do not. We elute these hybridized aptamers and, in phase II of selection, we combine them with a molecular target and re-expose the mixture to a fresh field of immobilized random 8 nucleotide oligonucleotides. In this case, we retain those sequences that did not hybridize to the immobilized nucleotides because we presume that they were constrained from doing so as a result of their binding to the target molecule.

This approach has worked well for the development of many aptamers for many targets, but for small targets, the approach implicitly drives selection for aptamers that bind at multiple sites to a given target molecule over those aptamers that bind the target more tightly. This is also an issue with SELEX selection for small molecules, as the immobilization of the target molecule also favours selection of aptamers that bind to more than the target molecule and hence have a higher probability of being retained in the selection process.

The need to increase the length of the aptamer in order to more adequately explore the structure space during the selection process as a function of the presence of primer recognition sequences increases the nature of this constraint on selection for small molecules with FRELEX or SELEX.

Here, we describe a synthetic library of aptamers in a closed sequence solution space, allowing a reproducible selection of aptamers, particularly useful for aptamer selection against small molecules.

### SUMMARY

The present invention relates to a synthetic library of aptamers with one module, said library comprising a plurality of aptamer oligonucleotide sequences, each comprising one module comprising at least two regions comprising a mixture of fixed and random nucleotides, two regions being interspersed with a stretch of fixed nucleotides, preferably with a stretch of adenosines and/or thymidines;
wherein internal sequence hybridization events are driven by variations in the random nucleotides of each of the at least two regions; and
wherein the aptamer oligonucleotide sequences comprise at most 11 random nucleotides so that the maximum number of possible different sequences in the library of aptamers is limited to 4 194 304; preferably at most 8 or 9 random nucleotides.

In some embodiments, each aptamer oligonucleotide sequence comprises at most 8 or 9 random nucleotides.

In some embodiments, each aptamer in the library of aptamers has the nucleotide sequence SEQ ID NO: 1, 7, 9, 17 or 18.

The present invention also relates to a synthetic library of aptamers, said library comprising a plurality of aptamer oligonucleotide sequences each comprising two or more modules, each of said modules comprising at least two regions comprising a mixture of fixed and random nucleotides, two regions being interspersed with a stretch of fixed nucleotides, preferably with a stretch of adenosines and/or thymidines;

wherein internal sequence hybridization events are driven by variations in the random nucleotides of each of the at least two regions within a same module or between two modules, preferably within a same module;
wherein each module comprises at most 11 random nucleotides, preferably at most 8 or 9 random nucleotides; and
wherein a restriction site is present between two modules.

In some embodiments, each aptamer in the library of aptamers has the nucleotide sequence SEQ ID NO: 6 or 14.

The present invention also relates to the use of the synthetic library of aptamers according to [0018]-[0020], in an aptamer selection process against a small target molecule, preferably wherein the small target molecule has a molecular weight below about 1 kDa.

The present invention also relates to a method of selecting aptamers that specifically bind to a small target molecule, preferably to a target molecule having a molecular weight below about 1 kDa, said method comprising contacting the small target molecule with the synthetic library of aptamers according to [0018]-[0020], and recovering the aptamer oligonucleotides that bound to the small target molecule; and optionally, contacting another small target molecule with the synthetic library of aptamers and recovering the aptamer oligonucleotides that did not bind to said other small target molecule.

In some embodiments, the small molecule is selected from the group consisting of antibiotics, volatile organic compounds (VOCs), amino acids, sugars, lipids, phenolic compounds, and alkaloids.

The present invention also relates to the use of the synthetic library of aptamers according to [0021]-[0022], in an aptamer selection process against a large target molecule, preferably wherein the large target molecule has a molecular weight above about 1 kDa.

The present invention also relates to a method of selecting aptamers that specifically bind to a large target molecule, preferably to a target molecule having a molecular weight above about 1 kDa, said method comprising contacting the large target molecule with the synthetic library of aptamers according to [0021]-[0022], and recovering the aptamer oligonucleotides that bound to the large target molecule; and optionally, contacting another large target molecule with the synthetic library of aptamers and recovering the aptamer oligonucleotides that did not bind to said other large target molecule.

In some embodiments common to all uses and methods disclosed herein, the selection in performed in a single round based on the statistical evaluation of the change in frequency of each sequence in the synthetic library of aptamers between a positive selection in the presence of the target molecule and a negative selection in the absence of the target molecule.

In some embodiments common to all uses and methods disclosed herein, the use or method is for selecting aptamers that specifically bind to a target molecule in a given 3-D or 4-D conformation.

In some embodiments common to all uses and methods disclosed herein, the use or method is for selecting aptamers that specifically bind to a full-length or native target molecule as opposed to the same target molecule that is degraded or otherwise cleaved.

In some embodiments common to all uses and methods disclosed herein, the use or method is for selecting aptamers that specifically bind to a target molecule as opposed to one or several counter-target molecules, wherein one or several positive selections are performed for both the target and the counter-target molecules with the same starting library, and aptamers that are preferentially selected in the presence of the target molecule compared to the counter-target molecules are recovered.

In some embodiments “a target molecule” refers to a known or unknown target molecule or a known or unknown target molecules.

In some embodiments “a counter-target molecule” refers to a known or unknown counter-target molecule or a known or unknown counter-target molecules.

In some embodiments the target molecule and/or the counter-target molecule is selected from the group consisting of antibiotics, volatile organic compounds (VOCs), amino acids, sugars, lipids, phenolic compounds, alkaloids, proteins and peptides, optionally extracellular domains of transmembrane receptors.

### DETAILED DESCRIPTION

The present invention relates to a synthetic library of aptamers comprising a plurality of aptamer oligonucleotide sequences, and related uses and methods.

The terms “aptamer” or “aptamer oligonucleotide” or “aptamer oligonucleotide sequence” refer to oligonucleotides that mimic antibodies in their ability to act as ligands and bind to a target molecule. In some embodiments, aptamers comprise natural or synthetic DNA nucleotides, natural or synthetic RNA nucleotides, modified DNA nucleotides, modified RNA nucleotides, or a combination thereof. The term “Library of aptamers” refer to DNA library of aptamers or RNA library of aptamers.

In some embodiments, the synthetic aptamer library comprises at least 50 000 different sequences, at least 75 000 different sequences, at least 100 000 different sequences, at least 250 000 different sequences, at least 500 000 different sequences, at least 750 000 different sequences, at least 1 000 000 different sequences, at least 1 250 000 different sequences, at least 1 500 000 different sequences, at least 1 750 000 different sequences, at least 2 000 000 different sequences, at least 2 250 000 different sequences, at least 2 500 000 different sequences, at least 2 750 000 different sequences, at least 3 000 000 different sequences, at least 3 250 000 different sequences, at least 3 500 000 different sequences, at least 3 750 000 different sequences, at least 4 000 000 different sequences, at least 4 250 000 different sequences.

In some embodiments, the synthetic aptamer library with one module comprises at most 4 194 304 different sequences, at most 1 048 576 different sequences, at most 262 144 different sequences or at most 65 536 different sequences.

In some embodiments, the synthetic aptamer library with two modules comprises at most 17.59218×10¹² different sequences, at most 1.09951×10¹² different sequences, at most 68 719 476 736 different sequences or at most 4 294 967 296 different sequences.

In some embodiment, the synthetic aptamer library comprises at least 50 000 aptamers, at least 100 000 aptamers, at least 500 000 aptamers, at least 1 000 000 aptamers, at least 2 000 000 aptamers, at least 3 000 000 different sequences, at least 4 000 000 aptamers, at least 5 000 000 aptamers, at least 6 000 000 aptamers, at least 7 000 000 aptamers, at least 8 000 000 aptamers, at least 9 000 000 aptamers, at least 10 000 000 aptamers, at least 20 000 000 aptamers, at least 30 000 000 aptamers, at least 40 000 000 aptamers, at least 50 000 000 aptamers, at least 60 000 000 aptamers, at least 70 000 000 aptamers, at least 80 000 000 aptamers, at least 90 000 000 aptamers, at least 100 000 000 aptamers, at least 200 000 000 aptamers, at least 300 000 000 aptamers, at least 400 000 000 aptamers, at least 500 000 000 aptamers, at least 600 000 000 aptamers, at least 700 000 000 aptamers, at least 800 000 000 aptamers, at least 900 000 000 aptamers, at least 1 000 000 000 aptamers, at least 2 000 000 000 aptamers, at least 3 000 000 000 aptamers, at least 4 000 000 000 aptamers, at least 5 000 000 000 aptamers, at least 6 000 000 000 aptamers, at least 7 000 000 000 aptamers, at least 8 000 000 000 aptamers, at least 9 000 000 000 aptamers, at least 10 000 000 000 aptamers, at least 20 000 000 000 aptamers, at least 30 000 000 000 aptamers, at least 40 000 000 000 aptamers, at least 50 000 000 000 aptamers, at least 60 000 000 000 aptamers, at least 70 000 000 000 aptamers, at least 80 000 000 000 aptamers, at least 90 000 000 000 aptamers, at least 100 000 000 000 aptamers, at least 200 000 000 000 aptamers, at least 300 000 000 000 aptamers, at least 400 000 000 000 aptamers, at least 500 000 000 000 aptamers, 700 000 000 000 aptamers, at least 600 000 000 000 aptamers, at least at least 800 000 000 000 aptamers, at least 900 000 000 000 aptamers, or at least 1 000 000 000 000 aptamers.

According to the invention, each aptamer oligonucleotide sequence in the library comprises a same modular structure, with at least one module, each of said module comprising a two regions interspersed with a stretch of fixed nucleotides.

The at least two regions comprise a mix of fixed nucleotides (i.e., nucleotides that do not vary in all the sequences of the library), and random nucleotides (i.e., nucleotides that vary in each sequence of the library).

In a preferred embodiment the fixed sequences are designed to minimize potential for complementary hybridization with other fixed sequences, both within a region and between regions. As such, all structural variation within the library is driven by variation in the identity of the random nucleotides and their ability to hybridize with other random or fixed nucleotides either within a region or between regions. In some embodiments, the at least two regions are at least partially complementary two by two. Depending on the nature of the random nucleotides in the at least two regions, they can be partially complementary or fully complementary. In some embodiments, two regions are therefore capable of forming a secondary structure element being a double-stranded stem, eventually with one or several mismatches when two random nucleotides do not hybridize. As such, all structural variations on the part of the aptamers are hence driven by variations in the random nucleotides, and their potential for hybridization between different regions (through their random nucleotides, or through their random and fixed nucleotides).

In some embodiments, each of the at least two regions comprises at least 3 nucleotides in total (fixed and random), such as 3, 4, 5, 6, 7 or more nucleotides in total. In some preferred embodiments, each of the at least two regions comprises 4 or 5 nucleotides in total (fixed and random).

In some embodiments, each of the at least two regions comprises from about 20% to about 80% of random nucleotides. In some embodiments, each of the at least two regions comprises at least 1, such as 1, 2, 3, 4, 5 or more random nucleotides.

In some preferred embodiments, each of the at least two regions comprises 4 or 5 nucleotides in total (fixed and random), among which 1, 2 or 3 random nucleotides.

In some preferred embodiment, each aptamer oligonucleotide sequence with one module in the library comprises at most 11 random nucleotides, so that the maximum number of possible different sequences in the library of aptamers is limited to 4¹¹=4 194 304. In some preferred embodiment, each aptamer oligonucleotide sequence with one module in the library comprises at most 10 random nucleotides, so that the maximum number of possible different sequences in the library of aptamers is limited to 4¹⁰=1 048 576. In some preferred embodiment, each aptamer oligonucleotide sequence with one module in the library comprises at most 9 random nucleotides, so that the maximum number of possible different sequences in the library of aptamers is limited to 4⁹=262 144. In some preferred embodiment, each aptamer oligonucleotide sequence with one in the library comprises at most 8 random nucleotides, so that the maximum number of possible different sequences in the library of aptamers is limited to 4⁸=65 536.

In some embodiments, the stretch(es) of fixed nucleotides comprise(s) adenosines and/or thymidines.

In some embodiments, the stretch(es) of fixed nucleotides comprise(s) at least 3 nucleotides or more, such as 3, 4, 5, 6, 7, 8, 9, 10 or more fixed nucleotides, preferably adenosines and/or thymidines. In some embodiments, the stretch(es) of fixed nucleotides comprise(s) a sufficient number of fixed nucleotides so that to be capable of forming a secondary structure element being a loop. Depending on the overall organization of the at least two regions and the stretch of fixed nucleotides, this loop can be a hairpin loops at the extremity of a double stranded stem, or interior loops between two double stranded stems.

Example 1 shows an exemplary embodiment wherein the aptamer oligonucleotide sequences comprise an overall A-s₁-B-s₂-C-s₃-D structure, wherein A, B, C and D are regions of fixed and random nucleotides and each of s₁, s₂ and s₃ is a stretch of fixed nucleotides. In this example, region A and region B can be at least partially complementary and form a stem, and regions B and region C can be at least partially complementary and form a stem; hence, the stretch s₂ would form a hairpin loop, and the stretches s₁ and s₃ together would form an internal loop.

An exemplary consensus sequence that can be shared by all the aptamers in the library is given as SEQ ID NO: 1, and further described in Example 1 below. It is to be understood that this consensus sequence is not intended to be a limiting feature of the present invention, and that the skilled artisan will readily contemplate modifications in this sequence, such as in the exact position and/or number of random nucleotides within the various regions, as well as in the nature, position and number of fixed nucleotides within the various regions and stretches.

In some embodiments, the consensus sequence is as set forth in SEQ ID NO: 34.

SEQ ID NO: 34
5′-AAANGWWWNNNGWWWCNNNWWWCNTTT-3′
wherein W indicate adenine or thymine; and N
indicates any nucleotide.

In some embodiments, the consensus sequence is as set forth in SEQ ID NO: 35.

SEQ ID NO: 35
5′-AAANGWWWNNNGWWWGNNNWWWCNTTT-3′
wherein W indicate adenine or thymine; and N
indicates any nucleotide.

In some embodiments, the consensus sequence is as set forth in SEQ ID NO: 36.

SEQ ID NO: 36
5′-AAANGWWWNNNCWWWCNNNWWWCNTAA-3′
wherein W indicate adenine or thymine; and N
indicates any nucleotide.

Also contemplated herein is a synthetic library of aptamers comprising a plurality of aptamer oligonucleotide sequences, and related uses and methods, wherein each aptamer oligonucleotide sequence in the library comprises two or more modules as described above, i.e., each module having at least two regions interspersed with a stretch of fixed nucleotides.

According to this embodiment, a restriction site may be present between each module, so as to allow individualization of each module by use of a restriction enzyme.

Two examples of restrictions enzymes are given in Examples 4 and 5 below:
the DraI enzyme, which recognizes and cleaves an AAA↓TTT sequence and produce blunt ends; and
the KasI enzyme, which recognizes and cleaves a G↓GCGCC sequence and produce overhangs.

It is however to be understood that these two enzymes are not intended to be a limiting feature of the present invention, and that the skilled artisan will readily contemplate using other restriction enzymes, depending on the specific nucleic acid sequence that is present at the interface between two modules.

Examples 4 and 5 show exemplary embodiments of such aptamer oligonucleotide sequences comprises two modules.

In some embodiments, the two or more modules can be identical. Alternatively, they can be different, e.g., they can differ by at least 1 fixed nucleotide, such as 1, 2, 3, 4, 5, 6, 7, 8, 9, 10 or more fixed nucleotides. When they differ, the at least two modules can find their differences in the fixed nucleotides of one or several regions, in the stretch(es) of fixed nucleotides, or in both.

In some embodiments, the aptamer oligonucleotide sequences comprise at least 2 modules as described above, such as at least 2, 3, 4, 5, 6, 7, 8 or more modules as described above. When the aptamer oligonucleotide sequences comprise more than 2 modules, such as 3 and more, the restriction site present between each module may be the same, or may be different; it is preferably the same.

The synthetic libraries of aptamers described herein find their use in several applications, as described in the Example section, and in particular in Example 6. The skilled artisan is familiar with techniques of aptamer selection, such as FRELEX or SELEX.

A particular application is a method of selecting aptamers that bind, preferably specifically bind, to a target molecule, which method comprises contacting a synthetic library of aptamers as described herein with the target molecule, and selecting those aptamer sequences that bind to the target molecule.

In particular, this method may be applied to the selection against a target molecule in multiple parallel applications of a single selection round. As detailed in the Example section, such selection in a single round is rendered possible and accurate with the synthetic libraries of aptamers described herein. Preferably, the basis for selection is the statistical evaluation of the change in frequency of each sequence in the library between a positive selection (i.e., in the presence of the target molecule) and a control, negative selection (i.e., in the absence of the target molecule).

The method can be applied, for instance, for the selection of aptamer sequences which bind specifically to a target molecule, but not to another specific molecule or group of molecules. In this case, the method will include a step of counter-selection against the other specific molecule or group of molecules.

The method can also be applied, for instance, for the selection of aptamer sequences that bind at relatively different affinities to different target molecules, e.g., for determining the presence of such molecules in a mixture and quantifying their relative amounts.

Such method can allow, for instance, to select aptamer sequences which bind specifically to a target molecule in a given 3-D or 4-D conformation, e.g., a target molecule in free state versus the same target molecule in complex with other molecules (4-D conformation); or a target molecule with a 3-D structure versus the same target molecule with a different folding.

Such method can also allow, for instance, to select aptamer sequences which bind specifically to a full-length or native target molecule versus the same target molecule which would have been degraded or otherwise cleaved into one or several fragments.

As explained in the Example section below, the synthetic library of aptamers, in particular when it comprises a single module, can be particularly useful for selecting aptamer sequences which bind specifically to small molecules. By “small molecule”, it is meant molecules which have a molecular weight below about 1 kDa.

Some examples of such small molecules include, without limitation, antibiotics, volatile organic compounds (VOCs), amino acids, some sugars, lipids, as well as phenolic compounds, alkaloids, and the like.

Conversely, the synthetic library of aptamers, in particular when it comprises two or more modules, can be particularly useful for selecting aptamer sequences which bind specifically to large or small molecules. By “large molecule”, it is meant molecules which have a molecular weight above about 1 kDa.

Some examples of such large molecules include, without limitation, proteins and polypeptides.

One object of the invention is an aptamer or aptamers obtainable from implementing the method of the invention.

Another object of the invention is the above-mentioned aptamer or aptamers or specific set of aptamers for use in a diagnostic or prognostic or disease monitoring method.

In one embodiment, the method as described above comprises contacting the aptamers or specific set of aptamers of the invention with a biological sample from at least one subject. In practice, the aptamers or specific set of aptamers of the invention would be applied on biological samples from a subject and would then be subjected to quantitative PCR analysis and the relative frequency of the aptamer sequences within the sample would be determined. In one embodiment, the relative frequency of diagnostic aptamers would be determined through a method other than quantitative PCR analysis, such as NGS analysis, hybridization to antisense sequences, or quantitative LCR analysis.

In one embodiment, the subject is/was diagnosed with the medical state, disease or condition under investigation. In one embodiment, the subject is at risk of developing the medical state, disease or condition under investigation. In one embodiment, the subject is/was not diagnosed with the medical state, disease or condition under investigation.

In one embodiment, the subject is/was diagnosed with the medical state, disease or condition under investigation. In one embodiment, the subject is at risk of developing the medical state, disease or condition under investigation. In one embodiment, the subject is/was not diagnosed with the medical state, disease or condition under investigation.

In one embodiment, the subject or diagnostic subject is a mammal, preferably a primate, more preferably a human. In one embodiment, the subject or diagnostic subject is a man. In one embodiment, the subject or diagnostic subject is a woman. In one embodiment, the subject or diagnostic subject is above the age of 20, preferably above the age of 30, 40, 50, 60, 70, 80, 90 years old or more. In one embodiment, the subject or diagnostic subject is from 30 to 90 years old, preferably from 40 to 90 years old, more preferably from 50 to 90 years old, even more preferably from 60 to 90 years old, even more preferably from 70 to 90 years old.

In one embodiment, the medical state, disease or condition include, but are not limited to, cancers, autoimmune diseases, cardiovascular diseases, infections, inflammatory diseases and metabolic diseases.

In one embodiment, the medical state, disease or condition include, but are not limited to, kidney disease, liver disease, inflammation or infections, dehydration, severe diarrhea, burns, malabsorption disorders, malnutrition, complications from diabetes, kidney failure, common variable immunodeficiency disorder (CVID), autoimmune disease, hepatitis, cirrhosis, chronic infections, or cancers.

In one embodiment of this invention, the above-mentioned aptamer or aptamers are for the identifying at least one aptamer for human serum albumin (HSA) or immunoglobulins (IgG).

In one embodiment the target molecule or molecules are contained in biological fluids, biological samples, or tissues.

In one embodiment Complex mixtures include, but are not limited to, blood serum, cerebrospinal fluid, urine, sweat, saliva, menstrual fluid, fecal suspensions, cell lysate suspensions, plant phloem fluid, and ground water.

Also disclosed herein are the following embodiments.

E1: A synthetic library of aptamers suitable for use in a method of selecting aptamers specifically binding to small molecules, said library comprising multiple regions composed of a mixture of fixed and random nucleotides designed in such a way that the random nucleotides have the potential to be homologous between specific regions and where these regions are separated by fixed sequences that enable hairpin turns between homologous regions contiguous, wherein the maximum number of possible different sequences is limited to 4 194 304.

E2: The synthetic library of aptamers according to E1, wherein the aptamer sequences comprise at most 11 nucleotides of random sequence, preferably at most 8 or 9 nucleotides of random sequence.

E3: The synthetic library of aptamers according to E1 or E2, wherein each aptamer in the library of aptamers has a sequence 5′-AAANGAAANNNGAAACNNNAAACNTTT-3′ with SEQ ID NO: 1, wherein N represents any nucleotide.

E4: The synthetic library of aptamers according to any one of E1 to E3, wherein small molecules are smaller in size than 1 000 Daltons.

E5: A method for the reproducible processing and analysis of aptamer selections for target molecules comprising a selection library of not more than 11 random nucleotides, and preferably 8 or 9 random nucleotides.

E6: The method according to E5, as applied to the selection of a target in multiple parallel applications of a single selection round where the basis for selection is the statistical evaluation of the change in frequency of each sequence in the library between the positive selection and a control selection with no target.

E7: The method according to E5 or E6, wherein the selection library is a synthetic library of aptamers according to any one of E1 to E4.

E8: The method according to any one of E5 to E7, for the identification of aptamers that bind specifically to one target molecule and not to another specific molecule or molecules.

E9: The method according to any one of E5 to E7, for the identification of aptamers that bind at relatively different affinities to different target molecules, for use in determining the presence of such molecules in a mixture and quantifying their relative amounts.

E10: The method according to any one of E5 to E9, wherein the target molecules are small molecules, preferably smaller in size than 1 000 Daltons.

E11: A synthetic library of aptamers composed of modules that can be separated post selection with a restriction enzyme, each of said modules comprising the design components as described in any one of E1 to E4.

E12: The synthetic library of aptamers according to E11, wherein each aptamer in the library of aptamers has a sequence 5′-AAANGAAANNNGAATGNNNAAACNTTTAAANGAAANNNCATTCNNNTTAC NTAA-3′ with SEQ ID NO: 6.

E13: Use of the library according to E11 or E12 in the method according to E5 or E6, said method being followed by the separation of each of the individual modules of the library by a restriction enzyme, wherein the target molecules are molecules larger than 1 000 Daltons.

E14: The use according to E13, for the selection of aptamers for more complex targets than molecules that are smaller than 1 000 Daltons in multiple parallel applications of a single selection round, where the basis for selection is the statistical evaluation of the change in frequency of each sequence in the library between the positive selection and a control selection with no target.

E15: The use according to E13 or E14, for the identification of aptamers that bind specifically to one molecule larger than 1 000 Daltons and not to another specific molecule or molecules.

E16: The according to E13 or E14, for the identification of the aptamers that bind to different molecules with different affinities for use in determining the presence of such molecules in a mixture, or for quantifying the relative amounts of such molecules.

E17: The according to E13 or E14, to characterize a difference between a molecule by itself, and the same molecule with another molecule bound to it.

E18: The according to E13 or E14, to characterize a difference in the manner in which a molecule is folded.

E19: The according to E13 or E14, to characterize different cleavage products of a protein.

E20: A synthetic library of aptamers, said library comprising a plurality of aptamer oligonucleotide sequences each comprising two or more modules, each of said modules comprising at least two regions comprising a mixture of fixed and random nucleotides, two regions being interspersed with a stretch of fixed nucleotides, preferably with a stretch of adenosines and/or thymidines;
wherein internal sequence hybridization events are driven by variations in the random nucleotides of each of the at least two regions within a same module or between two modules, preferably within a same module;
wherein each module comprises at most 11 random nucleotides, preferably at most 8 or 9 random nucleotides; and
wherein a restriction site is present between two modules.

E21: The synthetic library of aptamers according to E20, wherein each aptamer in the library of aptamers has the fixed nucleotides of the sequence SEQ ID NO: 6 or SEQ ID NO: 14, and varying sequence for the random nucleotides SEQ ID NO: 6 or SEQ ID NO: 14.

E22: Use of the synthetic library of aptamers according to E20 or E21, in an aptamer selection process against a target molecule.

E23: A method of selecting aptamers that specifically bind to a target molecule, wherein the method comprising contacting the target molecule with the synthetic library of aptamers according to E20 or E21, and recovering the aptamer oligonucleotides that bound to the target molecule; and optionally, contacting another target molecule with the synthetic library of aptamers and recovering the aptamer oligonucleotides that did not bind to said other target molecule.

E24: Use of the synthetic library of aptamers according to E20 or E21, to identify aptamers that bind to target molecules that differ in biological samples that are derived from individuals that differ in terms of phenotype where the identity of the target molecules that the aptamers bind to is not necessarily known.

E25: A method of selecting aptamers that specifically bind to a target molecule, wherein the method comprising contacting different biological samples that are derived from individuals that differ in terms of phenotype with the synthetic library of aptamers according to E20 or E21, and recovering the aptamer oligonucleotides that bound to the target molecules.

E26: The use or the method according to any one of E22 to E25, wherein the selection is performed in a single round or more selection rounds based on the statistical evaluation of the change in frequency of each sequence in the synthetic library of aptamers between a positive selection in the presence of the target molecule and a negative selection in the absence of the target molecule.

E27: The use or the method according to any one of E22 to E26, for selecting aptamers that specifically bind to a full-length or native target molecule as opposed to the same target molecule that is degraded, cleaved, or altered due to post-translational modifications, mutations, or changes in 3D structure.

E28: The use or the method according any one of E22 to E27, for selecting aptamers that specifically bind to a target molecule as opposed to one or several counter-target molecules, wherein one or several positive selections are performed for both the target and the counter-target molecules with the same library, and wherein preferably the aptamers are selected in the presence of the target molecule compared to the counter-target molecules.

E29: The use or the method according to any one of E22 to E28, wherein the target molecule and/or the counter-target molecule is selected from the group consisting of antibiotics, volatile organic compounds (VOCs), amino acids, sugars, lipids, phenolic compounds, alkaloids, proteins and peptides, optionally extracellular domains of transmembrane receptors.

E30: The use or the method according to any one of E22 to E29, wherein the target molecule or molecules are unknown.

E31: The use or the method according to any one of E22 to E30, wherein the target molecule or molecule are located on cell surfaces, wherein preferably the cell is a mammalian cell, bacterial cell, fungus, or virus, more preferably a mycobacterium cell or virus.

E32: The use or the method according to any one of E22 to E31, wherein the target or counter-target molecule or molecules are contained in biological fluids, biological samples, or tissues.

E33: The use or the method according to E32, wherein the biological fluids is a blood, plasma or serum.

E34: The use or the method according to any one of E22 to E33, for the identification of aptamers for target molecule comprising the following steps:
a. Performing aptamer selection on the desired target with the synthetic library of aptamers according to E20 or E21,
b. Performing selection on a counter-target or counter-targets with the same library either before, simultaneously or after step a,
c. Selecting the best performing aptamers on the desired target using statistical analysis, preferably using the formulation of a Z statistic for each sequence:

$$Z = \frac{Avg . freq . w / target - Avg . frequency w / o target}{Avg . of the standard deviations of the averages}$$

wherein “Avg. freq w/target” is the average frequency in the presence of selection for a target; and “Avg. freq w/o target” is the average frequency in the absence of selection for a target.
d. Optionally evaluating how these best performing aptamers on the desired target respond to selection on the counter-target or counter-targets by characterizing their response in the selection on a counter-target or counter targets with the same library, and
Selecting the aptamers by retaining only those sequences that do not exhibit a statistically significant response to selection to a counter-target or counter-targets,
or
Selecting aptamers that cross react to multiple targets, by retaining only those sequences that exhibit a statistically significant response to selection to counter-target or counter-targets.

E35: An aptamer or aptamers obtainable by the use or the method of any one of E22 to E34.

E36: An aptamer, wherein the aptamer has the nucleotide sequence of SEQ ID NO: 47 or SEQ ID NO: 48.

E37: Use of the synthetic library of aptamers according to E20 or E21, or the aptamer or aptamers according to claim E35 or E36 for the diagnostic of a disorder or disease.

E38: The use according to claim E37, wherein the disease or disorder is cancers, autoimmune diseases, cardiovascular diseases, infections, inflammatory diseases and metabolic diseases, optionally kidney disease, liver disease, inflammation or infections, dehydration, severe diarrhea, burns, malabsorption disorders, malnutrition, complications from diabetes, kidney failure, common variable immunodeficiency disorder (CVID), autoimmune disease, hepatitis, cirrhosis, chronic infections, or cancers.

E39: Use of the synthetic library of aptamers according to E20 or E21, or the aptamer or aptamers according to E35 or E36, for the detection and/or the quantification of a target molecule for diagnostic purpose.

E40: A method for identifying at least one aptamer against a target molecule, comprising the steps of:
a. Generating a synthetic library of aptamers according to E20 or E21,
b. Using SELEX or FRELEX selection process, incubating the candidate aptamers with said target,
c. Performing PCR reaction for each selected library,
d. Cutting the amplified library using a restriction enzyme that recognizes the restriction site designed to reside in the middle of the library, to divide the selected library into two different modules, Module A and Module B, with a difference in sequence at either the 5′ or 3′ end or both ends of the modules.
e. Dividing the restricted library into two aliquots and specifically amplifying Module A from one aliquot and Module B from the second aliquot with sufficient PCR reactions to ensure maintenance of an average copy number of at least 100 copies of each sequence.
f. Determining the frequency of each Module sequence within each selected library by dividing copy number by the total number of reads for that Module within each selected library.
g. Multiplying the frequency of the sequences in Module A by the frequency of the sequences in Module B thus creating a matrix of the frequencies of 4 294 967 296 possible sequences for each selection,
h. Performing steps b to g in at least duplicate to enable calculation of average frequencies and the standard deviation of these average frequencies for each 4 294 967 296 possible sequences for a given target and for selections with the same library in the absence of the target.
i. Identifying sequences in terms of determining the statistical significance of the average differences in frequencies between a library selected for a target and a library selected in the absence of the target, including but not limited to evaluating Z values by subtracting the average frequency of each sequence in the absence of target from the average frequency of each sequence in the presence of target, and dividing this subtracted value by the average of the standard deviation for the same sequence in both the presence and absence of the target.
j. Optionally, evaluating the binding performance of the sequences against the desired target and counter targets using methods from the group comprising: surface plasmon resonance imaging, isothermal titration calorimetry, dialysis, qPCR analysis of bound and unbound fractions of aptamer, electrochemical approaches, modulation of fluorescence either through quenching, or fluorescence polarization, lateral flow assays, ELISA assays, or HPLC analysis.

The present invention will be further understood from the following examples. However, the scope of protection of the present invention shall not be limited to these examples.

### BRIEF DESCRIPTION OF THE DRAWINGS

FIG. 1 shows the energy landscape of an 80-nucleotide oligonucleotide sequence. The number at the end of each vertical bar corresponds to a different predicted secondary structure of an oligonucleotide with the nucleic acid sequence SEQ ID NO: 37; the length of the vertical bars indicates the free energy required to change from one structure to another; the scale for these free energies is provided in the y-axis.

FIG. 2 shows the predicted proportion of secondary structures of the sequence referred to in FIG. 1 at equilibrium. In this figure, it was assumed that 100% of the sequence referred to in FIG. 1 was in the form of predicted “Structure 1”. The structures were allowed to evolve over time as a function of the energy landscape depicted in FIG. 1. It is clear that after 10³ time units, the system has equilibrated and the “Structure 2” is present at a higher proportion than “Structure 1”.

FIG. 3 is a dot plot showing the distribution of number of base pairs predicted across the 65 536 possible sequences derived from SEQ ID NO: 1.

FIG. 4 shows the annealing of double-stranded coupling oligonucleotides to a Neomer library.

FIG. 5 is an agarose gel showing the amplified Neomer library after selection and amplification, and prior to selection. Lane 1 is the molecular weight ladder containing oligonucleotides of length 766, 500, 350, 300, 250, 200, 150, 100, 75, 50 and 25 bp from top to bottom. The next three subsequent lanes are an amplified Neomer library after 4, 6 and 8 PCR cycles, respectively. The final three lanes are another amplified Neomer library at differing numbers of PCR cycles.

FIGS. 6A-B are two graphs showing the frequency distributions of all possible 65 536 sequences per module. FIG. 6A: positive selection against ampicillin; FIG. 6B: negative selection in absence of target.

FIG. 7 shows a matrix of the frequencies of 4 294 967 296 possible sequences.

FIG. 8 is a dot plot showing the top 10,000 sequences based on Z values on the x-axis, and fold-change between the positive and the negative selection on the y-axis.

FIG. 9 is the expected Z value distribution for the human serum albumin selection with a Neomer library based on a normal distribution with the observed mean and standard deviation of the Z values for all 4 294 967 296 sequences.

FIG. 10 is the observed frequency distribution of the top 10,000 sequences in terms of Z scores from the human serum albumin selection.

FIG. 11 is the observed distribution of Z scores in relation to Fold values for each of the top 10,000 sequences in terms of Z scores from the human serum albumin selection.

FIG. 12 is the observed frequency distribution of the top 10,000 sequences in terms of Z scores from the immunoglobulin (IgG) selection.

FIG. 13 is the observed distribution of Z scores in relation to Fold values for each of the top 10,000 sequences in terms of Z scores from the immunoglobulin (IgG) selection.

FIG. 14 is the observed distribution of Z scores in relation to Fold values for each of the top 10,000 sequences in terms of Z scores from the HSA selection in the IgG selection.

FIG. 15 is a chart of the resonance due to binding observed for aptamer fBHSA-6 selected for capacity to bind to human serum albumin and not to IgG in response to 250 nM concentration of human serum albumin (solid line) and 250 nM concentration of IgG (dashed line).

FIG. 16 is the observed distribution of Z scores in relation to Fold values for each of the top 10,000 sequences in terms of Z scores from the IgG selection in the HSA selection.

FIG. 17 is a chart of the resonance due to binding observed for aptamer fBIgG-2 selected for capacity to bind to IgG and not to HSA in response to 250 nM concentration of human serum albumin (dashed line) and 250 nM concentration of IgG (solid line).

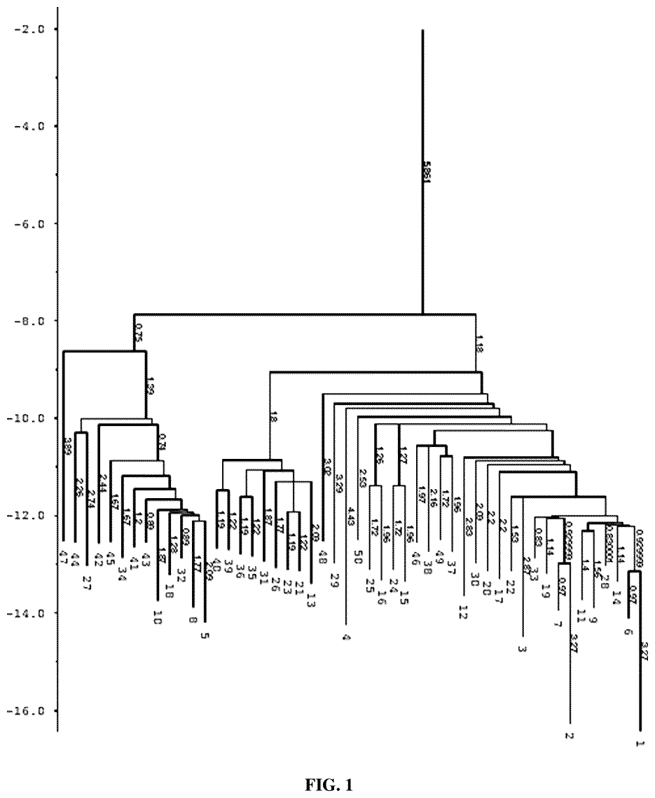

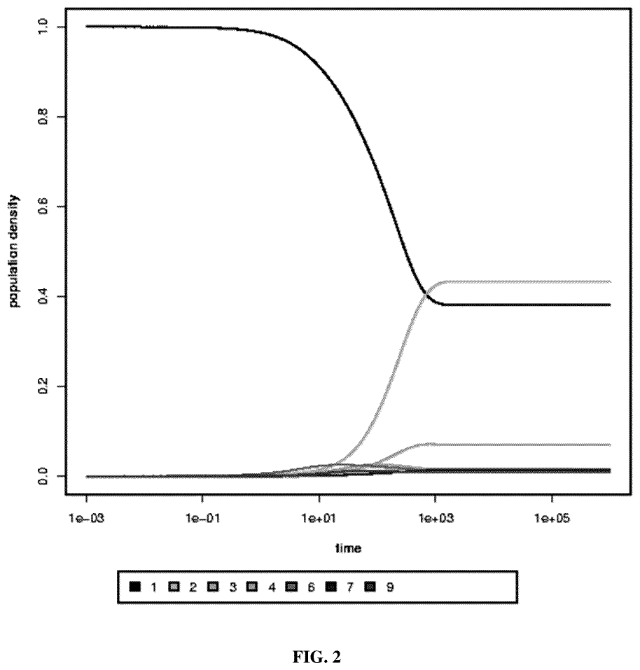

### EXAMPLES

### Example 1: Design of an Aptamer Library in which all Possible Sequences can be Characterized by Next-Generation Sequencing

U.S. Pat. No. 5,475,096 B2 was written prior to the development and wide-spread commercial application of next-generation sequencing. The examples provided within U.S. Pat. No. 5,475,096 B2 are based on the cloning of selected sequences into plasmids, transformation into bacteria and individual clone sequencing. The advent of next-generation sequencing has made this approach obsolete, with the possibility of millions of sequences being directly characterized from a single selection library following PCR amplification.

Given that this scale of sequence characterization was not available at the time this patent was filed, it is clear that the concept of limiting aptamer selection to a single selection depends entirely on the effectiveness of the selection process. In the present invention, we overcome this constraint by providing a method whereby the effectiveness of the selection process is not the limiting factor determining the necessary rounds of selection required. We achieved this by designing synthetic aptamer libraries with conserved elements and by including segments (or blocks) of random sequences that can be fully characterized by the current existing capacity of next-generation sequencing.

The number of possible sequences that can exist in a synthetic aptamer library can be calculated with the formula 4ⁿ, where “n” is the number of random oligonucleotides in the sequence. Table 1 provides the total number of possible sequences based on the total number of random nucleotides within a sequence of an oligonucleotide. Table 1 also provides the average number of copies expected of each sequence in an NGS analysis of 5×10⁶ sequences.

TABLE 1

| Number of random | Number of possible | Average copy number in NGS |
|---|---|---|
| nucleotides | sequences | analysis |
| 1 | 4 | 1250000 |
| 2 | 16 | 312500 |
| 3 | 64 | 78125 |
| 4 | 256 | 19531.25 |
| 5 | 1024 | 4882.8125 |
| 6 | 4096 | 1220.703125 |
| 7 | 16384 | 305.1757813 |
| 8 | 65536 | 76.29394531 |
| 9 | 262144 | 19.07348633 |
| 10 | 1048576 | 4.768371582 |
| 11 | 4194304 | 1.192092896 |
| 12 | 16777216 | 0.298023224 |
| 13 | 67108864 | 0.074505806 |
| 14 | 268435456 | 0.018626451 |
| 15 | 1073741824 | 0.004656613 |
| 16 | 4294967296 | 0.001164153 |
| 17 | 17179869184 | 0.000291038 |
| 18 | 68719476736 | 7.27596 × 10⁻⁵ |
| 19 | 2.74878 × 10¹¹ | 1.81899 × 10⁻⁵ |
| 20 | 1.09951 × 10¹² | 4.54747 × 10⁻⁶ |

It is clear from Table 1 that if the number of random nucleotides in a sequence exceeds 11, it is expected that, in an NGS analysis of 5 million sequences from such a library, not all sequences would be observed. Moreover, one trained in the art of evaluating NGS data of aptamer libraries would appreciate that there is variation in copy number in any naïve random library and as such, the probability of observing all possible sequences in a library would more likely be limited to libraries of 8 or 9 random nucleotides. As such, the use of 8 or 9 random nucleotides within a library would be a preferred embodiment of this invention.

We have also determined that the probability of an oligonucleotide composed of only 8 contiguous random nucleotides forming significant secondary structure is very low, as there is a need for the structure to contain a hairpin loop of at least 3 nucleotides between double-stranded regions. As such, to achieve the structural complexity necessary for aptamer function, it is necessary to intersperse the random nucleotides with defined, fixed sequences.

An example of a library with 8 random nucleotides interspersed with conserved elements is provided as SEQ ID NO: 1.

SEQ ID NO: 1
5′-AAANGAAANNNGAAACNNNAAACNTTT-3′
wherein N represents a random nucleotide.

This library has 8 random nucleotides and is specifically designed to maximize the effect of the random nucleotides on secondary structure, by placing the random nucleotides in four separate regions A to D as depicted below in bold underlined:

(SEQ ID NO: 1)
5′-AAANGAAANNNGAAACNNNAAACNTTT-3′
A B C D

The 4 regions A to D are composed of a mixture of fixed and random nucleotides. These regions are separated one from another by 3 A residues to provide capacity for hairpin turns and enable the necessary spatial freedom required for hairpin turns between possible hybridization of the regions. For instance, region C has the capacity to hybridize to region B, and region D has the capacity to hybridize to region A. All hybridization events within a single sequence must be anti-parallel with one strand running in a 5-to-3′ direction and the other in a 3-to-5′ direction.

The enablement of this example is not limited to this sequence, or this particular design. This example is provided to demonstrate that thoughtful consideration of the interspersion of fixed and random regions is necessary to optimize the capacity of the library to form an optimal number of secondary structures.

It has also been our experience that effective aptamers are composed of both hybridized regions (stems) and regions that are not hybridized (loops). Our library design takes this into consideration by optimizing the proportion of possible sequences that will have a mixture of stems and loops.

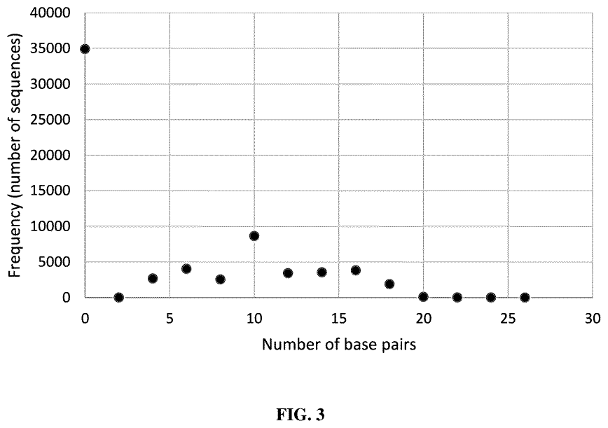

To confirm the potential efficacy of this library design, we tabulated all possible 65 536 sequences of the library of aptamers with one module with SEQ ID NO: 1, and determined the predicted secondary structures using batch LINUX commands in the RNAFold program (available at https://www.tbi.univie.ac.at/RNA/RNAfold.1.html). A distribution of shape complexity is provided in FIG. 3.

Overall, this means that 30 645 of the possible 65 536 sequences exhibit significant secondary structure in the absence of binding to another molecule. A preferred embodiment of this invention is a library design where over 25% of all possible sequences form secondary structure. It is recognized by the Inventor that the library of this example may not provide sufficiently complex structures to bind to relatively complex targets such as proteins: as such, a preferred embodiment would be a library of aptamers, each aptamer comprising at least one module, with for each module from 8 to 11 random nucleotides. Such library would be suitable for selection against small molecules (e.g., with a molecular weight below about 1 kDa) or against any larger targets exhibiting only few charged groups.

### Example 2: A Method for the Preparation of a Library without Primer Recognition Sequences for Next Generation Sequence Analysis

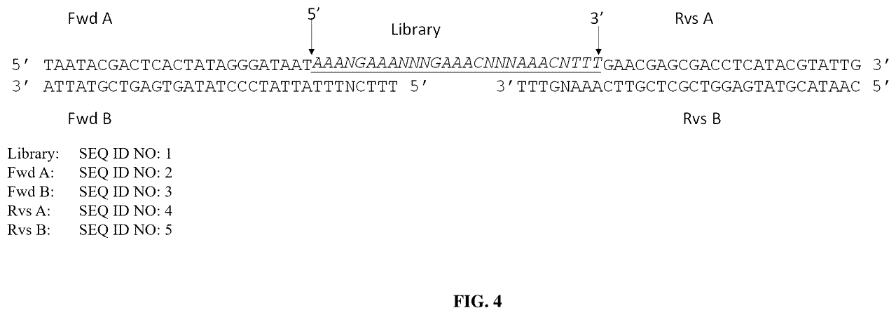

The library of Example 1 is an example of a library enabling the present invention that does not have primer recognition sequences. A method describing how such a library would be prepared for next-generation sequence analysis is provided below and illustrated in FIG. 4.

Step a): the library is synthesized with a 5′ phosphate group to enable effective ligation.

Step b): two double-stranded coupling oligonucleotides are designed to facilitate preparation of the library for NGS.

Step c): sequences Fwd A and Fwd B are annealed with each other and the sequences Rvs A and Rvs B are annealed to each other.

The library of Example 1 is an example of a library enabling the present invention that does not have primer recognition sequences. A method describing how such a library would be prepared for next-generation sequence analysis is provided below and illustrated in FIG. 4.

The four sequences in this example provided for the double-stranded coupling oligonucleotides are as follows:

-Fwd A
SEQ ID NO: 2
5′-TAATACGACTCACTATAGGGATAAT-3′
-Fwd B
SEQ ID NO: 3
5′-TTTCNTTTATTATCCCTATAGTGAGTCTATTA-3′
-Rvs A
SEQ ID NO: 4
5′-GAACGAGCGACCTCATACGTATTG-3′
-Rvs B
SEQ ID NO: 5
5′-CAATACGTATGAGGTCGCTCGTTCAAANGTTT-3′

The Rvs B sequence can be labeled at its 5′ end, for instance with a biotin moiety; the Rvs A oligonucleotide is preferably synthesized with a 5′ phosphate.

Step d): the double-stranded coupling constructs are annealed with the library sequences.

Step e): the construct created in step d) is used to add primer recognition regions to the library, using, e.g., a T4 DNA ligase. This creates a post-selection library. Only the sense strand containing the library is efficiently ligated, as the antisense strands are separated by a gap in the hybridized construct.

Step f): the post-selection library is washed from all extraneous sequences; for instance, when the Rvs B sequence is labelled with biotin, the entire construct can be coupled to immobilized streptavidin (e.g., streptavidin coated resin, streptavidin agarose, streptavidin coated magnetic beads, etc.).

Step g): the sense strand of the post-selection library is recovered, e.g., from the immobilized streptavidin using an elution step. Preferably, this step is performed in absence of any chemical denaturing agents to avoid additional steps of washing. For instance, elution can be performed using heat alone.

Step h): The sense strand of the post-selection library is amplified with appropriate primers for preparation for NGS.

It is a preferred embodiment of this invention that both the naïve (i.e., unselected) library and a selected library be prepared in the same manner, for instance according to the method described above. This would then enable a reliable comparison of the frequency of each sequence in the naïve library to its frequency in the selected library. Indeed, in SELEX or FRELEX, candidate aptamers are chosen from the selection process based on their overall enrichment in terms of frequency across selection rounds; however, if only a single round of selection is performed, then the selection is based on the sequences with the highest frequency which may be a biased choice if one doesn't know their frequency in the naïve library.

In the present invention, candidate sequences are chosen based on the relative proportion of their increase or decrease in frequency in the selected library from their frequency in the naïve library. This is the sole basis for their selection.

Moreover, a key advantage of the present invention is that, given we are characterizing the frequency of all possible sequences in the naïve and selected libraries, the selection process is reasonably expected to be reproducible. This means that replications of the same selection process should result in similar changes in overall frequency for the selected sequences. As such, the design of the library described in Example 1, and the processing of such libraries for NGS, establishes a basis for an innovative approach to defining the optimal aptamers from a single-round selection process. This is described in more detail in Example 3 below.

### Example 3: A Method for the Statistical Analysis of Replicated Single Step Selection Processes for the Identification of Optimal Aptamers for any Given Target

It was an insight of the Inventor that, given that libraries could be designed where a significant proportion of the possible sequences would exhibit secondary and tertiary structures, and given that the frequencies of all sequences within such libraries could be characterized, this meant that the aptamer selection process is reproducible. As such, it is possible to design aptamer identification processes that are based on statistical analysis of replicated selection experiments using the same libraries. This capacity has not previously existed within the field of aptamer selection.

An example of selection strategy is as follows.

Step a): a sample of the library containing 1010 copies is combined with a target and allowed to incubate in binding buffer for preferably 5 minutes, although incubation time could be longer or shorter.

Step b): the mixture from step a) is applied to a gold surface coated with immobilized oligonucleotides that are composed of 8 contiguous random nucleotides (i.e., a FRELEX field as described in EP 3 344 805 B1 or U.S. Pat. No. 10,415,034 B2), and allowed to incubate.

Step c): the supernatant from this surface containing those sequence that have not hybridized to the immobilized random 8 nucleotide oligonucleotides is recovered.

The product of step c) constitutes a “selected library”. This would then be directly processed for NGS as described in Example 2.

It is a preferred enablement of this new approach to characterize the frequency of each sequence in the naïve (unselected) library from at least two iterations (replications) of independent and separate single-step selection processes, as described above, but in the absence of any target molecule contacted with the library. A selection for aptamers in the presence of a target molecule would be performed in the same manner, with at least two separate and independent iterations of a single selection round.

The application of such a process will result in average frequencies and standard deviations of these frequencies of all sequences in a library. For example, if 8 random nucleotides are used in the selected library (as per the example of SEQ ID NO: 1), then we would have average expected frequencies and standard deviations of these average frequencies for each of all possible 65 536 sequences in the absence of selection, and in the presence of selection for a specific target.

This information allows evaluation of aptamer sequences that exhibit the highest statistically significant deviation, either in terms of increased or decreased frequency, in the presence of selection for the target versus in the absence of the target. An example of the processing of such data would involve the use of the following formula well-known in statistics, as the formulation of a Z statistic for each sequence:

$$Z = \frac{Avg . freq . w / target - Avg . frequency w / o target}{Avg . of the standard deviations of the averages}$$

wherein “Avg. freq w/target” is the average frequency in the presence of selection for a target; and “Avg. freq w/o target” is the average frequency in the absence of selection for a target.

This statistical analysis process for the identification of optimal aptamers from the selection process is not limited to only a positive/negative comparison. It is a further insight of the Inventor that, given the reproducibility of the process, positive selection could be applied against one or more counter-targets and sequences identified that differ between selections. This leads to implicit identification of sequences with desired specificity for one target over another.

It is a further insight of the Inventor that such a process is not limited to qualitative differences defined as binding or not binding. The effect of different targets on the relative frequencies of aptamers within a defined library as described herein could also be use to identify aptamers that have relatively different binding affinities to multiple targets. Such aptamers could then be used individually, or in combination to quantify the relative amounts of several different target molecules in a mixture.

### Example 4: Extension of Structural Complexity of Aptamer Libraries without Loss of Reproducibility

It was noted previously that the structural complexity of the library of Example 1 may be limiting when considering aptamer selection for complex targets (e.g., molecules with a molecular weight above 1 kDa or with several charged groups), such as proteins. In this invention, this limitation can be overcome by increasing the potential for structural complexity while maintaining the reproducibility of this aptamer selection process through the introduction of multiple modules in each aptamer of the library.

This multiple library modules, similar to the library described in Example 1, are synthesized in a library as contiguous units that remain together for selection, but can be cleaved into the respective modules prior to NGS characterization. This is shown below, with an exemplary sequence of SEQ ID NO: 6 comprising SEQ ID NO: 9 as an exemplary sequence of a first module (example of Module A) and a similar sequence of SEQ ID NO: 7 an exemplary sequence of a second module (example of Module B).

SEQ ID NO: 6
5′-AAANGAAANNNGAATGNNNAAACNTTTAAANGAAANNNCATTCNNN
TTACNTAA-3′
wherein N represents a random nucleotide.
An example of Module A,
SEQ ID NO: 9
5′-AAANGAAANNNGAATGNNNAAACNTTT-3′
An example of Module B,
SEQ ID NO: 7
5′-AAANGAAANNNCATTCNNNTTACNTAA-3′

This library has 16 random nucleotides, each aptamer being divided into two modules A and B, and each module being sub-divided in four separate regions as depicted below in bold underlined, from 5′ to 3′:

```
                  Module A Module B
AAANGAAANNNGAATGNNNAAACNTTT AAANGAAANNNCATTCNNNTTACNTAA
    A B C′ D A B′ C D′
```

In this library design, when compared to the single-module design with SEQ ID NO: 1 of Example 1, region C was modified in Module A to start with a G nucleotide (named region C′), and region B was modified in Module B to end with a C nucleotide (named region B′). These changes were made to ensure that the sequenced products of the two modules could be differentiated from each other.

Region D was also modified in Module B (named region D′) to enable separate amplification of this module. This difference enables the option of preparing Module B from Module A separately with different hex codes in the forward primer for NGS.

These changes also ensure that all potential for hybridization be majorly driven by the random nucleotides and not by the fixed nucleotides. Indeed, any implicit secondary structure due to hybridization of complementary fixed nucleotides would reduce the potential diversity of the solution space.

This library of aptamers, comprising in this example two modules (Module A and Module B), can be synthesized and used as a contiguous strand in selection. Prior to NGS preparation, the library can be cleaved into the two modules with the use of a restriction enzyme (in this particular example, for instance, a DraI enzyme which recognizes and cleaves an AAA↓TTT sequence and produce blunt ends). The use of this enzyme, or any other restriction enzyme, can be facilitated by the addition of an antisense oligonucleotide, for instance with SEQ ID NO: 8, that would hybridize to the library astride Module A and Module B, creating a double-stranded recognition site for the restriction enzyme.

SEQ ID NO: 8
5′-TTTCNTTTAAANGTTT-3′
AAANGAAANNNGAATGNNNAAACNTTT↓AAANGAAANNNCATTCNNNTTACNTAA
TTTGNAAA↑TTTNCTTT

The remaining free antisense fragments can be easily removed using, e.g., a primer cleanup column.

The retained library modules can then be processed separately for NGS analysis. Each library module comprises 65 536 possible sequences and the library as a whole comprises 4 294 967 296 possible sequences.

As such, the concept of the selection process comprising a closed sequence solution space remains satisfied. Samples of the library for selection or for naïve library processing for NGS analysis would be made with at 4 294 967 296 sequences, such that each possible sequence would have an average of 100 copies in the naïve library.

The frequency of the random nucleotides within each position can be determined for each module separately with confidence by NGS. The product of the frequency for each position is equivalent to the frequency of each possible sequence. Thus, the frequency of each of the possible 4 294 967 296 sequences could be determined by the product of the individual frequencies of nucleotide identity at each random nucleotide position.

As such, the concept of enabling reproducible aptamer selection articulated in Examples 1 to 3 is retained. We have demonstrated that all of the possible sequences in the naïve library would be present in each aliquot of the library analyzed, and that the frequency of each possible sequence could be ascertained in the naïve library and in the selected libraries.

We have the capacity to characterize the frequency of each of the 65 536 sequences present in each module, thus we have the capacity to characterize the frequency of each of the 4 294 967 296 sequences in a library of aptamers with SEQ ID NO: 6. As such, the reproducibility of the selection process is maintained, while the range of complexity in potential secondary and tertiary structures is increased.

In theory, this concept could be expanded to include more than 16 random nucleotides and thus, more than 4 294 967 296 possible sequences. As the sequence length is increased however, the number of individual sequences that are not observed in the original library would decrease simply as a function of sampling. In this case, we would increase the original sample of aptamers sequences applied to a target from 4 294 967 296 sequences to a larger number, to ensure adequate coverage of all sequences.

It is also possible to vary the number of random nucleotides to any possible number as long as the number within a given library module can be fully characterized by NGS.

It is also possible to vary the library structure, by changing the sequence identity and/or the length of the fixed sequence nucleotides.

Following digestion of SEQ ID NO: 6 with DraI, the two Modules A and B are produced, with SEQ ID NOs: 9 and 7, respectively. These modules require a redesign of the sequences for preparation for next generation sequencing as follows.

-Fwd B
SEQ ID NO: 10
5′-TTTCNTTTATTATCCCTATAGTGAGTCGTATTA-3′
-Rvs A
SEQ ID NO: 11
5′-GAACGAGCGACCTCATACGTATTTG-3′
-Rvs B module A
SEQ ID NO: 12
5′-CAAATACGTATGAGGTCGCTCGTTCAAANGTTT-3′
-Rvs B module B
SEQ ID NO: 13
5′-CAAATACGTATGAGGTCGCTCGTTCTTANGTAA-3′

Similarly to Example 2, the Rvs B sequences with SEQ ID NOs: 12 and 13 can be labeled at their 5′ end, for instance with a biotin moiety; and the Rvs A oligonucleotide with SEQ ID NO: 11 is preferably synthesized with a 5′ phosphate.

These would be used to prepare each module for NGS analysis as described for the single module in Example 2. This preparation could either be done simultaneously or with separate aliquots of the digested library.

### Example 5: A Simplified Library Design for Neomer Selection

A potential difficulty encountered with the practical application of the sequences and approach described in Example 4 could be inefficiency, as the process requires the ligation of primer recognition regions to a library sequence prior to amplification. In practice, the ligation step may be difficult to robustly reproduce, resulting in an arbitrary loss of a proportion of the sequences, and/or mis-amplification of the library with non-flanking primer sequences. This difficulty can be overcome with the use of a library with SEQ ID NO: 14.

SEQ ID NO: 14
5′-CCAGATACAGACNNGAGGNNNGAATNNNAACCATCGGCGCCAACAN
NNCATTCNNNCAGANNCAGTAGACAGC-3′

In this example, SEQ ID NO: 14 is first amplified with a forward primer with SEQ ID NO: 15 and a reverse primer with SEQ ID NO: 16. The amplified sequence is then restricted with the restriction enzyme KasI forming two restricted fragments: an example of Module A with SEQ ID NO: 17 and an example of Module B with SEQ ID NO: 18. Module A is amplified for NGS analysis with a forward NGS sequence containing a hex code (SEQ ID NO: 19) and a reverse NGS sequence (SEQ ID NO: 20). A second round of amplification is performed with a forward NGS 2 primer containing a hex code (SEQ ID NO: 21) and a reverse NGS 2 primer (SEQ ID NO: 22).

SEQ ID NO: 15
5′-CAAATACGTATGAGGTCGCTCGTTCCCAGATACAGAC-3′
SEQ ID NO: 16
5′-TAATACGACTCACTATAGGGATAATGCTGTCTACTG-3′
SEQ ID NO: 17
5′-CAAATACGTATGAGGTCGCTCGTTCCCAGATACAGACNNGAGGNNNGAAT
NNNAACCATCG-3′
SEQ ID NO: 18
5′-GCGCCAACANNNCATTCNNNCAGANNCAGTAGACAGCATTATCCCTATAG
TGAGTCGTATTA-3′
SEQ ID NO: 38
5′-CCCTACACGACGCTCTTCCGATCTATCACGCAAATACGTATGAGGTCGCTC
GTTC-3′
an example of module A,
SEQ ID NO: 20
5′-GGTCAGACGTGTGCTCTTCCGATCGGGGCGCCGATGGTT-3′
SEQ ID NO: 39
5′-AATGATACGGCGACCACCGAGATCTACACTCTTTCCCTACACGACGCTCTT
CCG-3′
SEQ ID NO: 40
5′-CAAGCAGAAGACGGCATACGAGATGTGACTGGAGTTCAGACGTGTGCTCT
TCC-3′
an example of Fwd NGS 1 module B,
SEQ ID NO: 41
5′-CCCTACACGACGCTCTTCCGATCTATCACGGCGCCAACA-3′
an example of Rev NGS 1 module B,
SEQ ID NO: 42
5′-GGTCAGACGTGTGCTCTTCCGATCGGGTAATACGACTCACTATAGGGATAA
TGCTGTCTACTG-3′
Underlined in SEQ ID NO: 38 is the hex code sequence (ATCACG), which varies
with different libraries.

### Example 6: Selection of Aptamers that Bind to Antibiotics

The Neomer library described in Example 6 was applied to the selection of the antibiotics ampicillin and amoxicillin, using the FRELEX selection process as described in EP 3 344 805 B1 or U.S. Pat. No. 10,415,034 B2.

FRELEX requires the preparation of an immobilization field consisting of a gold chip coated with thiolated random 8-base pair DNA oligonucleotides. The 8-mer thiolated random oligonucleotides were dissolved in 50 μL of phosphate buffer saline (PBS) (8.0 mM Na₂PO₄, 1.4 mM KH₂PO₄, 136 mM NaCl, 2.7 mM KCl, pH 7.4) at a concentration of 10 μM. This solution was incubated at room temperature for 1 hour on gold surface chip (7×10×0.3 mm; Xantec, Germany). The chip was then air-dried; 50 μL of a solution containing thiol-terminated polyethylene glycol (SH-PEG) molecules was added and incubated for 30 min at room temperature with gentle shaking. This step blocks any remaining gold surface that is not covered with 8-mers. SH-PEG was subsequently added a second time for 16 hours. After that, the SH-PEG solution was discarded from the chip and the functionalized gold chip surface was washed with deionized water and air-dried.

In the first step of FRELEX, 2×10¹³ sequences from the random aptamer library described previously were snap-cooled by heating the library to 95° C. for 10 minutes followed by immediate immersion in ice bath. These single-stranded DNA sequences were incubated with the desired target in 50 μL of selection buffer (10 mM Tris, 120 mM NaCl, 5 mM MgCl₂, 5 mM KCl) for 30 minutes at room temperature. This solution was then applied to the functionalized immobilization field (gold chip with 8-mers) for 15 minutes at room temperature. The solution was removed and kept. The immobilization field was washed twice with 50 μL of 1× selection buffer. These solutions were pooled and purified using the oligonucleotide clean-up protocol of the Monarch PCR & DNA Clean-Up kit, as described by NEB, and eluted with 400 μL of deionized water.

After this selection, PCR was used to amplify the selected single-stranded DNA into double-stranded DNA for an appropriate number of cycles to create a clear band of approximately 5 ng of amplified DNA. All PCR procedures were carried out according to standard molecular biology protocols and under the following conditions: 95° C. for 5 minutes, 4 cycles at 95° C. for 10 seconds, 35° C. for 15 seconds, 72° C. for 30 seconds, 4 cycles at 95° C. for 10 seconds, 64° C. for 15 seconds and 72° C. for 30 seconds, followed by a final extension at 72° C. for 5 minutes.

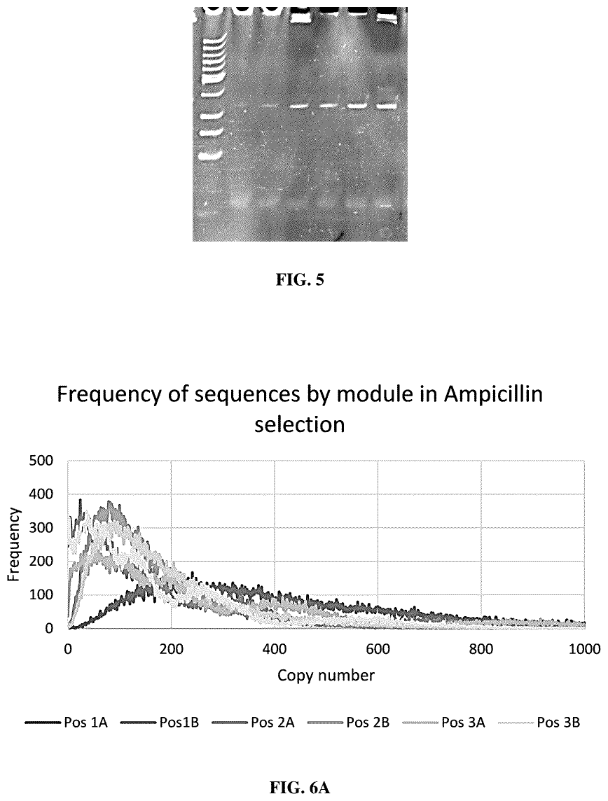

FIG. 5 shows the amplified Neomer library after selection and amplification, and prior to selection.

Four PCR reactions of 50 μL each were performed at the optimum number of PCR cycles for each selected library. The Neomer library from each of these reactions was then purified using an oligonucleotide clean-up protocol (Monarch PCR & DNA Clean-Up kit, NEB) and eluted into 40 μL of water. This purified product was restricted with KasI restriction enzyme (NEB), according to NEB protocol for this enzyme.

Following restriction, the solution contained both “Module A” and “Module B”. Half of this solution was used to amplify Module A for NGS analysis and the other half was used for Module B. Each product was diluted six-fold prior to amplification.

Throughout the selected library processing to NGS analysis, it is important to maintain the average copy number of the sequences above 100. We achieved this by increasing the number of PCR reactions for each step as a function of the PCR cycles. The amplified product was purified using the Monarch PCR & DNA Clean-Up kit.

All libraries were sequenced using Illumina NovaSeq at the TCAG facility (Hospital for Sick Children, Toronto, Canada). Fastq files obtained upon sequencing were converted into a Fasta format, and the copy number of each of the possible 65 536 sequences in each of Module A and Module B was characterized using a proprietary Python script that we have developed for this application. The frequency of each Module sequence was determined by dividing copy number by the total number of reads for that Module.

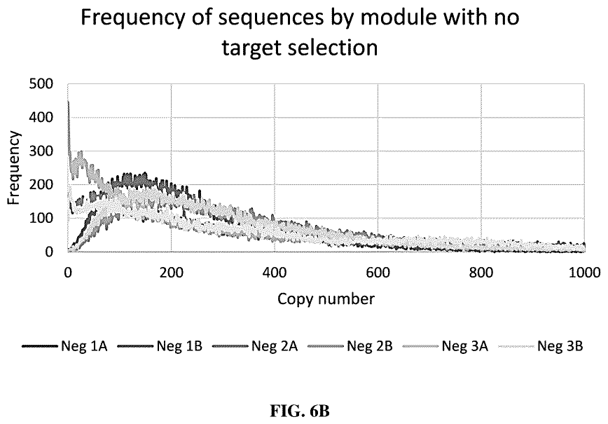

FIG. 6 provides the frequency in copy number of each of the 65 536 sequences in each Module for three replicates of the positive ampicillin selection (FIG. 6A), and three replicates of a negative (i.e., without target) selection (FIG. 6B).

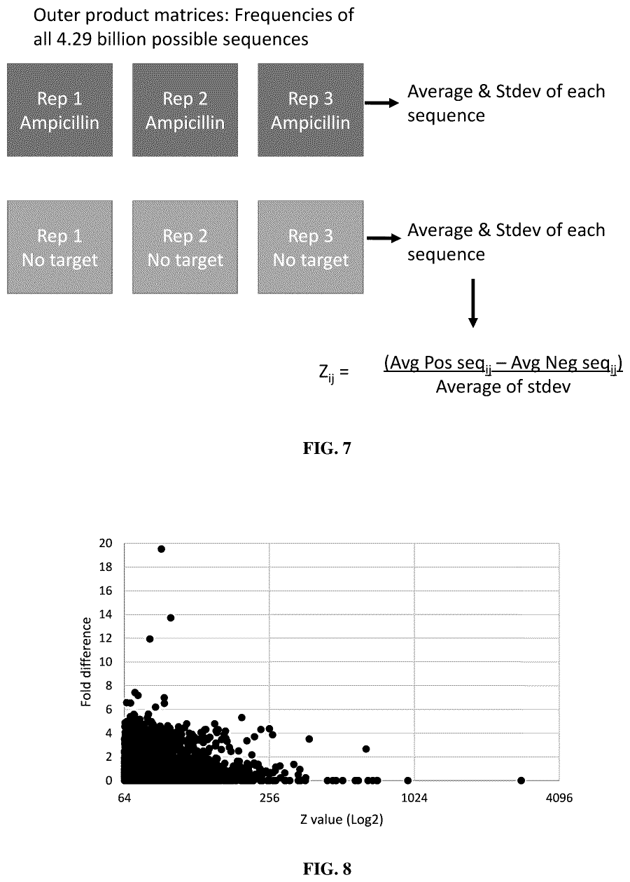

These frequencies were multiplied with each other, thus creating a matrix of the frequencies of 4 294 967 296 possible sequences for each selection. This was evaluated for the identification of candidate sequences as shown in FIG. 7.

We then identify the top 10 000 sequences in terms of Z values. However, relying solely on a Z value can lead to bias, in that the Z value can be very high because of very low standard deviations rather than because of a large difference between positive and negative selection. We compensated for this by plotting the top 10 000 sequences based on Z values on one axis, and by fold-change between the positive and the negative selection on the other axis (FIG. 8).

The top 11 candidate sequences in terms of both Z values and fold change were selected for binding assays (Table 2).

TABLE 2

| Aptamer name | SEQ ID NO | Z value | Fold change |
|---|---|---|---|
| Amp-13 | 23 | 645.0136 | 2.661603 |
| Amp-26 | 24 | 374.6329 | 3.510966 |
| Amp-55 | 25 | 263.931 | 3.861203 |
| Amp-60 | 26 | 255.821 | 4.36379 |
| Amp-72 | 27 | 236.0355 | 4.306906 |
| Amp-126 | 28 | 196.3811 | 5.317402 |
| Amp-1130 | 29 | 99.69954 | 13.71249 |
| Amp-1352 | 30 | 93.80888 | 6.51017 |
| Amp-1355 | 31 | 93.66725 | 6.981253 |
| Amp-1503 | 32 | 91.14148 | 19.51418 |
| Amp-2216 | 33 | 81.64084 | 11.926 |

SEQ ID NO: 23
5′-CCAGATACAGACCGGAGGTCTGAATCTCAACCATCGGCGCCAACATT
TCATTCAACCAGAACCAGTAGACAGC-3′
SEQ ID NO: 24
5′-CCAGATACAGACACGAGGTCTGAATCCTAACCATCGGCGCCAACAG
CTCATTCGCCCAGACCCAGTAGACAGC-3′
SEQ ID NO: 25
5′-CCAGATACAGACCTGAGGTCTGAATCTTAACCATCGGCGCCAACAAC
GCATTCTGCCAGAGTCAGTAGACAGC-3′
SEQ ID NO: 26
5′-CCAGATACAGACCGGAGGATCGAATCTAAACCATCGGCGCCAACAGC
GCATTCCTCCAGACGCAGTAGACAGC-3′
SEQ ID NO: 27
5′-CCAGATACAGACCTGAGGTCGGAATCCAAACCATCGGCGCCAACAGC
GCATTCCTCCAGACGCAGTAGACAGC-3′
SEQ ID NO: 28
5′-CCAGATACAGACTCGAGGTCTGAATCTGAACCATCGGCGCCAACACC
GCATTCATTCAGACACAGTAGACAGC-3′
SEQ ID NO: 29
5′-CCAGATACAGACCGGAGGTCTGAATCCAAACCATCGGCGCCAACAGC
GCATTCCTCCAGACGCAGTAGACAGC-3′
SEQ ID NO: 30
5′-CCAGATACAGACCCGAGGCCTGAATCCCAACCATCGGCGCCAACAGC
GCATTCCTCCAGACGCAGTAGACAGC-3′
SEQ ID NO: 31
5′-CCAGATACAGACCAGAGGTCTGAATCCCAACCATCGGCGCCAACAGG
GCATTCTGGCAGACTCAGTAGACAGC-3′
SEQ ID NO: 32
5′-CCAGATACAGACCGGAGGTCTGAATCTCAACCATCGGCGCCAACAGC
GCATTCTAGCAGATACAGTAGACAGC-3′
SEQ ID NO: 33
5′-CCAGATACAGACCAGAGGTCTGAATCGTAACCATCGGCGCCAACAGC
GCATTCCTCCAGACGCAGTAGACAGC-3′

### Example 7: Possible Applications of this Invention

The examples that follow should be considered as possible applications of the invention, and not a comprehensive list of all possible applications.

### Epitope Mapping

It is recognized by the Inventor that the innovation detailed in this invention by reducing aptamer selection to a reproducible process would enable the mapping of all possible epitopes on target molecules. With traditional aptamer selection, either SELEX or FRELEX, it is not possible to determine whether two different aptamer sequences bind to the same epitope on a given molecular target or not.

For example, a traditional aptamer selection against a protein could lead to the identification of many aptamers that bind to said protein. Examination of the sequences of the aptamers and of their secondary structures does not provide us with an insight into whether the aptamer binds to the same epitope on the protein or not.

It is common in the art of aptamer selection to consider an aptamer sequence as a string of characters. A change of one character from another is referred to as a Hamming distance of 1. The same change and a change at a different position would be considered as a Hamming distance of 2. Given that we have characterized all possible sequences within a selected library for their capacity to bind to a given target we can then characterize the nature of this binding as a function of Hamming distances from the island of sequences that bind. This knowledge provides us with a basis for predicting what is important about the structure of an aptamer in 3-D space as it relates to a specific target molecule.

As such, we can specify different structural solutions to the problem of target binding, and we can establish which of these structural solutions overlap with each other and which do not.

This capacity has profound potential as a tool in diagnostics and therapeutics. For example, it would be possible to compare a complete aptamer library response map to a specific protein with and without the presence of therapeutic that binds to it. This could be used to characterize the nature of the binding event between the therapeutic and the target protein, and to quantify the amount of therapeutic bound.

A similar approach can be contemplated regarding the characterization of all possible cleavage events of a protein. Maps of reproducible aptamer library responses to all possible peptides could be constructed and used to create subsets of aptamers for the identification of all possible outcomes. One such application could be an extension of the capacity to identify cleavage products from cardiac infractions in brain natriuretic peptides and troponin.

This method could be used to identify the fingerprint of any target molecule on a defined aptamer library. By fingerprint, I mean the effect of a given target on the individual frequencies of each possible sequence in the library. This extends beyond the strategy of identifying specific proteins by developing a large library of aptamers with each aptamer specific to a different target protein. This means identifying specific target proteins with a single round of selection against a defined, closed sequence space aptamer library, and correlating the effect of this selection on the effect of known sequences.

As such, the effect of any given protein is implicitly the cumulative effect of each of its epitopes. By screening for different aptamer frequency effects across different proteins it will be possible to identify specific epitopes that are responsible for each effect.

### DNA Mismatches

A double stranded oligonucleotide could have a single position mismatch in nucleotides. There are seven such possible mismatches (as a combination of G with T does not constitute a significant mismatch). It is clear that a library such as the ones described in the examples above, or a different library but adhering to the same principles of a closed sequence solution space would provide an effective means of selecting aptamers for binding to such specific mismatches.

The sequence of the library could be designed so as to avoid potential for hybridization to the surrounding regions used for the identification of the mismatch.

### Example 8: Application of the Neomer Library for the Identification of Aptamers for Human Serum Albumin (HSA) and Immunoglobulins (IgG)

The proteins human serum albumin (HSA) and a pool of immunoglobulin antibodies (IgG) were applied in triplicate in selection against the library of Example 6. The methods of application with FRELEX selection was applied, and the products were characterized by NGS analysis. Statistical analysis was carried out as described in Example 3. This included creating frequency matrices for each of the replicates for the protein selected and a negative control buffer for each of 4 294 967 296 sequences, determining averages and standard deviations for each of these sequences. These averages and standard deviations were then used to define Z scores for each sequence on the following basis.

(Average of sequence in selection against protein−average of sequence in selection against buffer)/average of standard deviation of sequence in selection against protein and buffer

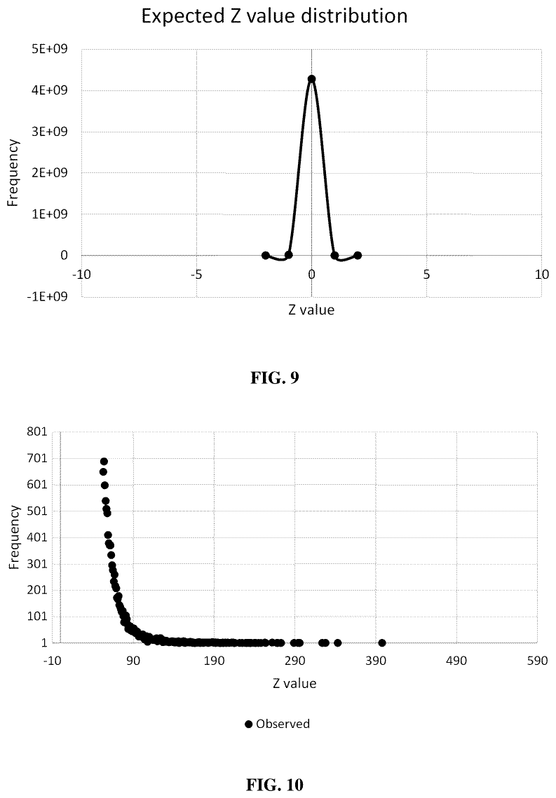

The overall average for the Z scores for the HSA selection was-0.57 with a standard deviation of 0.16. This means that if the data was normally distributed the distribution would appear as provided in FIG. 9.

The observed frequency distribution of the top 10,000 sequences appears as described in FIG. 10.

This comparison confirms the statistical significance of each of these 10,000 sequences in relation to binding to HSA.

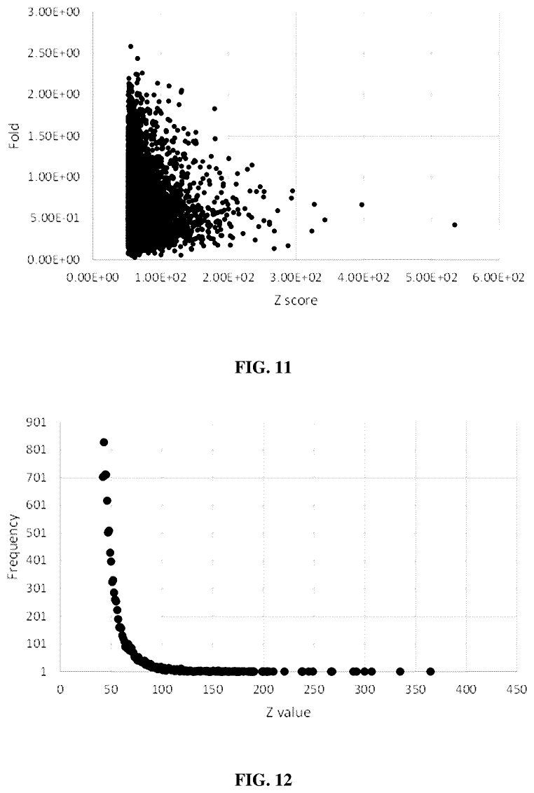

We also determined the fold value differences for each of these top 10,000 sequences. The fold value is the observed average frequency of the sequence in selection against HSA divided by the average frequency of the same sequence in selection against buffer. We subtract unity from this dividend to clearly show the direction of the fold effect (FIG. 11).

IgG selection was performed in an identical manner.

The overall mean for the IgG selection was −0.49 and the standard deviation was 0.085. This means that the expected normal distribution of these values in the absence of selection would be even closer to the mean than with the HSA selection.

The distribution of the Z scores for the IgG selection is shown in FIG. 12.

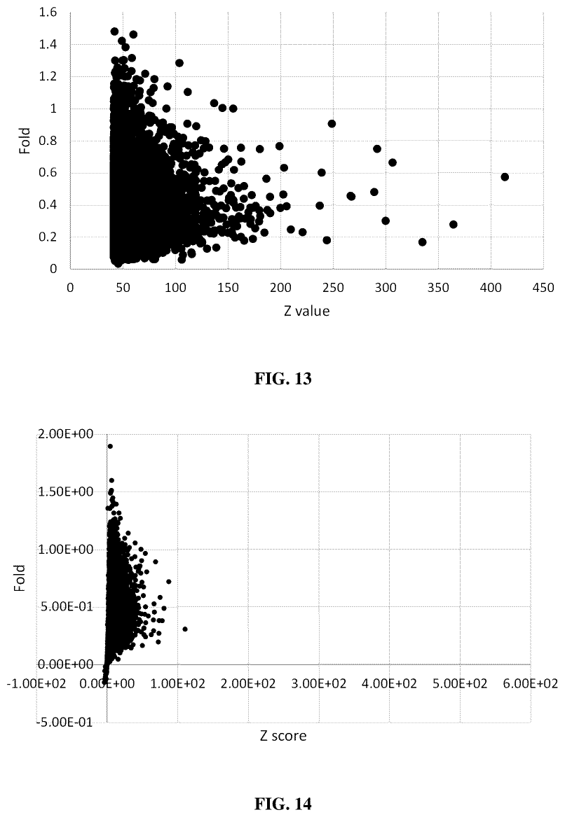

The distribution of the sequences in terms of Z value and Fold value is provided in FIG. 13.

All of these 10,000 sequences selected for either HSA or IgG are expected to bind to their respective target. In the next example we will describe a further step, the analysis of these sequences for cross-reactivity to other proteins as a step to refine candidate sequence selection for binding assays.

### Example 9: Screening Cross-Reaction of Selected Aptamers for a Given Target Against a Counter Target

An implicit advantage that antibody selection has over aptamer selection is the presence of the immune tolerance system underlying antibody selection. This means that any antibody that binds to an epitope that is found in the same organism is eliminated prior to production. A similar system does not exist for aptamer selection. It is possible to perform counter selection against other targets, but the effectiveness of counter selection is a function of the strength of the cross-reactive binding. The stronger the cross-reactivity to another target the more effectively sequences will be removed or diminished in abundance.

The difficulty that arises as a result of the observed lack of an immune tolerance system in aptamer selection is the difference in the abundance of certain molecules in biological fluids versus others. Human serum albumin is present on average at a concentration of 600 uM in blood and IgGs are present at a concentration that is similar. If a targeted protein is present at an abundance of 600 pM this means that there are 1 billion more molecules of HSA present compared to the target protein. Even if an aptamer binds to HSA with a thousand fold less affinity than to the target molecule, the abundance of the HSA molecules will saturate binding of the aptamer such that no significant binding to the target molecule is observed.

This clearly is a significant constraint to the development of aptamers for commercial applications in diagnosis or therapeutics. In this example we will describe how the Neomer aptamer selection process can be used in silico to mimic immune tolerance and provide more effective screening of candidate sequences for their cross-reactivity to other proteins.

First, we will describe how we screen the top 10,000 sequences selected for human serum albumin (HSA) for their potential to cross-react (bind) to immunoglobulin (IgG).

We determine the frequency of each of the possible 65,536 sequences within each module (A and B) for the selection of HSA versus buffer and for the selection of IgG versus buffer. We evaluate the frequency of each of the 10,000 HSA sequences identified above in the IgG selection by determining their frequency within each module and replicate for the IgG selection versus buffer. For example, the top HSA sequence from the selection above (this is represented by the point on the Z score axis where the value is greater than 500) is:

SEQ ID NO: 43
5′-CCAGATACAGACTTGAGGTTTGAATACGAACCATCGGCGCCAACATT
ACATTCCCACAGATCCAGTAGACAGC-3′

This consists of an example of module A sequence of SEQ ID NO: 44

5′-CCAGATACAGACTTGAGGTTTGAATACGAACCATCG-3′

And an example of module B sequence of SEQ ID NO: 45

5′-GCGCCAACATTACATTCCCACAGATCCAGTAGACAGC-3′

We can evaluate the frequency of both the module A and module B sequences in each of the replicates for IgG selection. The buffer comparison frequencies are the same between both selections. This product of the frequencies for each module correspond to the frequency for the entire Neomer sequence in the IgG selection.

The distribution of the top 10,000 HSA sequences described in terms of Z score and fold value are provided in the same terms in FIG. 14 in regard to how they performed in the IgG selection.

It is clear that the Z scores were much less for these sequences in response to IgG selection than they were for HSA selection which is reasonable given that these sequences were selected as the best performers in response to HSA selection (Table 3).

We selected the following sequences from this comparative analysis as candidate sequences for binding assays.

TABLE 3
Comparison of Z scores and fold values for selected
Aptamers in performance against HSA and IgG.

| | HSA | | IgG | |
|---|---|---|---|---|
| | Z score | Fold | Z score | Fold |
| fBHSA-1 | 534.6732 | 0.419807 | 5.687703 | 0.338036 |
| fBHSA-2 | 397.2445 | 0.665289 | 14.93829 | 0.349889 |
| fBHSA-6 | 295.1485 | 0.832639 | 9.948514 | 0.522505 |
| fBHSA-11 | 267.7119 | 0.135036 | 0.786504 | 0.032988 |
| fBHSA-106 | 161.5022 | 0.277642 | 0.101903 | 0.005523 |
| fBHSA-130 | 152.3724 | 0.2559 | 0.334868 | 0.005868 |
| fBHSA-4435 | 63.75173 | 0.334501 | −1.50918 | −0.08779 |
| fBHSA-4951 | 62.14766 | 0.299803 | −1.14905 | −0.04628 |

These sequences were synthesized with a 5′ thiol and immobilized onto a gold surface in triplicate spots at a concentration of 5 uM in a volume of 10 nL. A negative aptamer of the same length not selected for binding to serum albumin was also spotted on the gold chips in the same manner. These were allowed to dry, and the remainder of the surface was blocked with thiolylated polyethylene glycol with an average molecular weight of 5 kda. The gold chips were then inserted on top of a glass prism in a Horiba Openplex Surface Plasmon Resonance imaging instrument (SPRi). Human serum albumin at two concentrations (100 nM and 250 nM) was injected over the chip at a volume of 200 uL and a flow rate of 50 uL/min. We also injected a pool of immunoglobulin (IgG) at a concentration of 250 nM at the same volume and flow rates.

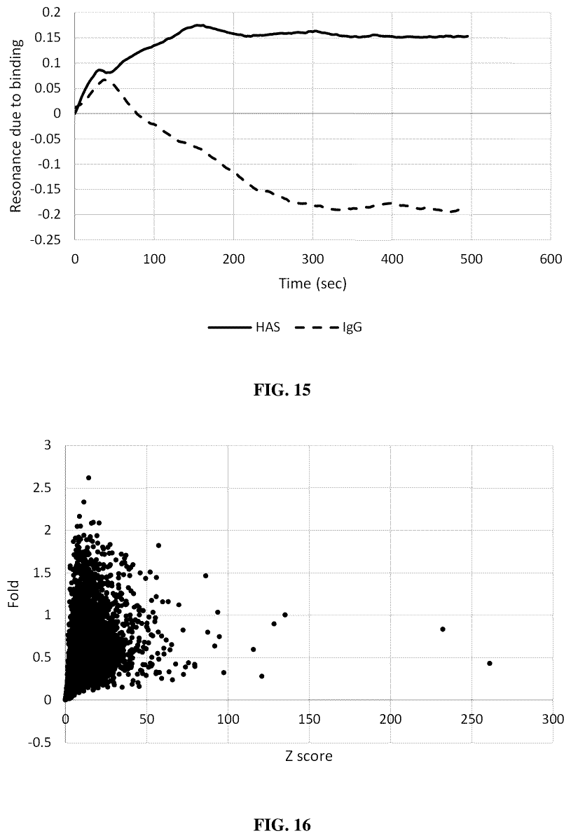

Total resonance was measured for each spot and averaged across spots for a given aptamer. Resonance due to binding was calculated by subtracting the total resonance observed for the negative aptamer from each of the candidate sequences. This is shown in FIG. 15.

Given a flow rate of 50 uL/min and an injection volume of 200 uL, we expect the association phase (that portion of the experimental data where the protein is flowing over the aptamers) to last a maximum of 240 seconds and the disassociation phase (that portion of the experimental data where the protein is no longer flowing over the aptamers) to begin immediately after the association phase.

We calculate the kd for each aptamer by numerically solving the following equation;

x′˜−kd*x
where:
x′=the derivative of the resonance due to binding in the disassociation curve,
x=the resonance value, and
kd=the rate of complex breakdown

We use the mathematical coding language R to identify the solution for kd that best fits the map between the derivative of the resonance values and the actual resonance values.

With this estimate for kd we are now ready to calculate the value for ka (or kon). For this we use the following formula;

x′˜ka*c*Rmax−(ka*c+kd)*x
where:
ka=the rate of complex formation,
c=the concentration of the analyte,
Rmax=the maximum resonance value observed,
kd=the rate of complex breakdown, and
x=the resonance value.

The binding coefficients calculated for the aptamer fBHSA-6 was:

$$k d = 1.29 E - 3 ,$$

$$ka = 2.19 E 5 , and$$

$$kD = 5.89 E - 9 .$$

Sequence of fBHSA-6, SEQ ID NO: 46

5′-CCAGATACAGACCAGAGGCGGGAATAAGAACCATCGGCGCCAACA
CCACATTCATTCAGATCCAGTAGACAGC-3′

The same process was repeated for the top 10,000 IgG sequences in terms of their performance against HSA. The FIG. 16 provides the Z value and fold values for these top 10,000 IgG sequences as they performed in the HSA selection.

The candidate sequences chosen for binding analysis are described in the Table 4 below.

TABLE 4

| | IgG | | HSA | |
|---|---|---|---|---|
| | Z value | Fold | Z value | Fold |
| fBIgG-2 | 3.64E+02 | 2.77E−01 | 1.77E+00 | 1.58E−01 |
| fBIgG-3 | 3.35E+02 | 1.67E−01 | 1.43E+00 | 1.41E−01 |
| fBIgG-25 | 1.85E+02 | 2.27E−01 | −1.18E−02 | −3.58E−04 |
| fBIgG-35 | 1.72E+02 | 3.10E−01 | 7.22E−01 | 5.09E−02 |
| fBIgG-1435 | 6.80E+01 | 3.36E−01 | −7.56E+00 | −9.54E−02 |
| fBIgG-4512 | 5.02E+01 | 4.11E−01 | −4.10E−01 | −6.31E−02 |
| fBIgG-3510 | 5.35E+01 | 1.04E−01 | −1.93E−02 | −8.13E−04 |

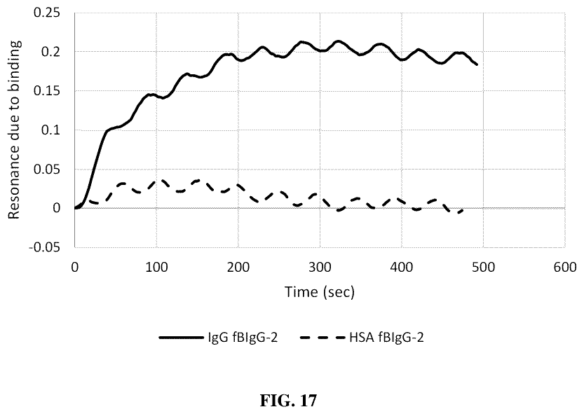

Binding analysis for the selected IgG sequences was performed in a manner identical to that described for the HSA sequences. The performance of one of the selected aptamer sequences in surface plasmon resonance imaging analysis is provided in FIG. 17.

The predicted binding affinity coefficients for this aptamer fBIgG-2 against IgG were

$$k d = 7.56 E - 03 ,$$

$$ka = 3.69 E + 05 , and$$

$$kD = 2.05 E - 08 .$$

Sequence of fBIgG-2, SEQ ID NO: 47

5′-CCAGATACAGACCCGAGGTGAGAATTTCAACCATCGGCGCCAACA
AAACATTCTCTCAGATACAGTAGACAGC-3′

The following nucleotide sequences are listed herein, from 5′ to 3′:

SEQ ID NO: 1:
AAANGAAANNNGAAACNNNAAACNTTT
SEQ ID NO: 2:
TAATACGACTCACTATAGGGATAAT
SEQ ID NO: 3:
TTTCNTTTATTATCCCTATAGTGAGTCTATTA
SEQ ID NO: 4:
GAACGAGCGACCTCATACGTATTG
SEQ ID NO: 5:
CAATACGTATGAGGTCGCTCGTTCAAANGTTT
SEQ ID NO: 6:
AAANGAAANN NGAATGNNNA AACNTTTAAA NGAAANNNCA
TTCNNNTTAC NTAA
SEQ ID NO: 7:
AAANGAAANN NCATTCNNNT TACNTAA
SEQ ID NO: 8:
TTTCNTTTAA ANGTTT
SEQ ID NO: 9:
AAANGAAANN NGAATGNNNA AACNTTT
SEQ ID NO: 10:
TTTCNTTTAT TATCCCTATA GTGAGTCGTA TTA
SEQ ID NO: 11:
GAACGAGCGA CCTCATACGT ATTTG
SEQ ID NO: 12:
CAAATACGTA TGAGGTCGCT CGTTCAAANG TTT
SEQ ID NO: 13:
CAAATACGTA TGAGGTCGCT CGTTCTTANG TAA
SEQ ID NO: 14:
CCAGATACAG ACNNGAGGNN NGAATNNNAA CCATCGGCGC
CAACANNNCA TTCNNNCAGA NNCAGTAGAC AGC
SEQ ID NO: 15:
CAAATACGTA TGAGGTCGCT CGTTCCCAGA TACAGAC
SEQ ID NO: 16:
TAATACGACT CACTATAGGG ATAATGCTGT CTACTG
SEQ ID NO: 17:
CAAATACGTA TGAGGTCGCT CGTTCCCAGA TACAGACNNG
AGGNNNGAAT NNNAACCATC G
SEQ ID NO: 18:
GCGCCAACAN NNCATTCNNN CAGANNCAGT AGACAGCATT
ATCCCTATAG TGAGTCGTAT TA
SEQ ID NO: 20:
GGTCAGACGTGTGCTCTTCCGATCGGGGCGCCGATGGT
SEQ ID NO: 23:
CCAGATACAG ACCGGAGGTC TGAATCTCAA CCATCGGCGC
CAACATTTCA TTCAACCAGA ACCAGTAGAC AGC
SEQ ID NO: 24:
CCAGATACAG ACACGAGGTC TGAATCCTAA CCATCGGCGC
CAACAGCTCA TTCGCCCAGA CCCAGTAGAC AGC
SEQ ID NO: 25:
CCAGATACAG ACCTGAGGTC TGAATCTTAA CCATCGGCGC
CAACAACGCA TTCTGCCAGA GTCAGTAGAC AGC
SEQ ID NO: 26:
CCAGATACAG ACCGGAGGAT CGAATCTAAA CCATCGGCGC
CAACAGCGCA TTCCTCCAGA CGCAGTAGAC AGC
SEQ ID NO: 27: CCAGATACAG ACCTGAGGTC GGAATCCAAA
CCATCGGCGC CAACAGCGCA TTCCTCCAGA CGCAGTAGAC AGC
SEQ ID NO: 28:
CCAGATACAG ACTCGAGGTC TGAATCTGAA CCATCGGCGC
CAACACCGCA TTCATTCAGA CACAGTAGAC AGC
SEQ ID NO: 29:
CCAGATACAG ACCGGAGGTC TGAATCCAAA CCATCGGCGC
CAACAGCGCA TTCCTCCAGA CGCAGTAGAC AGC
SEQ ID NO: 30:
CCAGATACAG ACCCGAGGCC TGAATCCCAA CCATCGGCGC
CAACAGCGCA TTCCTCCAGA CGCAGTAGAC AGC
SEQ ID NO: 31:
CCAGATACAG ACCAGAGGTC TGAATCCCAA CCATCGGCGC
CAACAGGGCA TTCTGGCAGA CTCAGTAGAC AGC
SEQ ID NO: 32:
CCAGATACAG ACCGGAGGTC TGAATCTCAA CCATCGGCGC
CAACAGCGCA TTCTAGCAGA TACAGTAGAC AGC
SEQ ID NO: 33:
CCAGATACAG ACCAGAGGTC TGAATCGTAA CCATCGGCGC
CAACAGCGCA TTCCTCCAGA CGCAGTAGAC AGC
SEQ ID NO: 34:
AAANGWWWNN NGWWWCNNNW WWCNTTT
SEQ ID NO: 35:
AAANGWWWNN NGWWWGNNNW WWCNTTT
SEQ ID NO: 36:
AAANGWWWNN NCWWWCNNNW WWCNTAA
SEQ ID NO: 37:
AACTCGCGAG CCACGGGTAG TCCTCCACCT CACTAGGGGG
TTGGCGGATC TCAGCTGACAAAACAAGATC TTTGCGATCG
SEQ ID NO: 38:
CCCTACACGA CGCTCTTCCG ATCTATCACG CAAATACGTA
TGAGGTCGC TCGTT
SEQ ID NO: 39:
AATGATACGG CGACCACCGA GATCTACACT CTTTCCCTAC
ACGACGCTCT TCCG
SEQ ID NO: 40:
CAAGCAGAAG ACGGCATACG AGATGTGACT GGAGTTCAGA
CGTGTGCTCT TCC
SEQ ID NO: 41:
CCCTACACGA CGCTCTTCCG ATCTATCACG GCGCCAACA
SEQ ID NO: 42:
GGTCAGACGT GTGCTCTTCC GATCGGGTAA TACGACTCAC
TATAGGGATA ATGCTGTCTA CTG
SEQ ID NO: 43:
CCAGATACAG ACTTGAGGTT TGAATACGAA CCATCGGCGC
CAACATTACA TTCCCACAGA TCCAGTAGAC AGC
SEQ ID NO: 44:
CCAGATACAG ACTTGAGGTT TGAATACGAA CCATCG
SEQ ID NO: 45:
GCGCCAACAT TACATTCCCA CAGATCCAGT AGACAGC
SEQ ID NO: 46:
CCAGATACAG ACCAGAGGCG GGAATAAGAA CCATCGGCGC
CAACACCACA TTCATTCAGA TCCAGTAGAC AGC
SEQ ID NO: 47:
CCAGATACAG ACCCGAGGTG AGAATTTCAA CCATCGGCGC
CAACAAAACA TTCTCTCAGA TACAGTAGAC AGC

Wherein W indicate adenine or thymine; and N indicates any nucleotide.

1-20. (canceled)

21. A synthetic library of aptamers, said library comprising a plurality of aptamer oligonucleotide sequences each comprising two or more modules, each of said modules comprising at least two regions comprising a mixture of fixed and random nucleotides, two regions being interspersed with a stretch of fixed nucleotides;
wherein internal sequence hybridization events are driven by variations in the random nucleotides of each of the at least two regions within a same module or between two modules;
wherein each module comprises at most 11 random nucleotides; and
wherein a restriction site is present between two modules.

22. The synthetic library of aptamers according to claim 21, wherein each aptamer in the library of aptamers has the fixed nucleotides of the sequence SEQ ID NO: 6 or SEQ ID NO: 14, and varying sequence for the random nucleotides SEQ ID NO: 6 or SEQ ID NO: 14.

23. An aptamer selection process against a target molecule using a synthetic library of aptamers, comprising selecting aptamers from the synthetic library of aptamers that bind with the target molecule, said library comprising a plurality of aptamer oligonucleotide sequences each comprising two or more modules, each of said modules comprising at least two regions comprising a mixture of fixed and random nucleotides, two regions being interspersed with a stretch of fixed nucleotides;
wherein internal sequence hybridization events are driven by variations in the random nucleotides of each of the at least two regions within a same module or between two modules;
wherein each module comprises at most 11 random nucleotides; and
wherein a restriction site is present between two modules.

24. A method of selecting aptamers that specifically bind to a target molecule, wherein the method comprising contacting the target molecule with the synthetic library of aptamers according to claim 21, and recovering the aptamer oligonucleotides that bound to the target molecule.

25. A method of selecting aptamers that specifically bind to a target molecule, wherein the method comprising contacting different biological samples that are derived from individuals that differ in terms of phenotype with the synthetic library of aptamers according to claim 21, and recovering the aptamer oligonucleotides that bound to the target molecules.

26. The process according to claim 23, wherein the selection is performed in a single round or more selection rounds based on the statistical evaluation of the change in frequency of each sequence in the synthetic library of aptamers between a positive selection in the presence of the target molecule and a negative selection in the absence of the target molecule.

27. The process according to claim 23, for selecting aptamers that specifically bind to a full-length or native target molecule as opposed to the same target molecule that is degraded, cleaved, or altered due to post-translational modifications, mutations, or changes in 3D structure.

28. The process according to claim 23, for selecting aptamers that specifically bind to a target molecule as opposed to one or several counter-target molecules, wherein one or several positive selections are performed for both the target and the counter-target molecules with the same library.

29. The process according to claim 23, wherein the target molecule and/or the counter-target molecule is selected from the group consisting of antibiotics, volatile organic compounds (VOCs), amino acids, sugars, lipids, phenolic compounds, alkaloids, proteins and peptides; or wherein the target molecule or molecules are unknown.

30. The process according to claim 23, wherein the target molecule is located on cell surfaces.

31. The process according to claim 23, wherein the target is contained in biological fluids, biological samples, or tissues.

32. An aptamer selection process against a target molecule, using a synthetic library of aptamers, for the identification of aptamers for target molecule comprising the following steps:
a) performing aptamer selection on the desired target with the synthetic library of aptamers according to claim 21,
b) performing selection on a counter-target or counter-targets with the same library either before, simultaneously or after step a),
c) selecting the best performing aptamers on the desired target using statistical analysis,
and
selecting the aptamers by retaining only those sequences that do not exhibit a statistically significant response to selection to a counter-target or counter-targets,
or
selecting aptamers that cross react to multiple targets, by retaining only those sequences that exhibit a statistically significant response to selection to counter-target or counter-targets.

33. An aptamer or aptamers obtainable by the process according to claim 32.

34. An aptamer, wherein the aptamer has the nucleotide sequence of SEQ ID NO: 47 or SEQ ID NO: 48.

35. A method of treating a disorder or a disease in a subject in need thereof, comprising the steps of: i) diagnosing the disorder or the disease using the synthetic library of aptamers according to claim 21; and ii) treating the subject being diagnosed during the step i).

36. A method for the detection and/or the quantification of a target molecule, comprising contacting a biological sample with at least one aptamer selected from the synthetic library of aptamers according to claim 21.

37. A method for identifying at least one aptamer against a target molecule, comprising the steps of:
a) generating a synthetic library of aptamers according to claim 21,
b) using SELEX or FRELEX selection process, incubating the candidate aptamers with said target,
c) performing PCR reaction for each selected library,
d) cutting the amplified library using a restriction enzyme that recognizes the restriction site designed to reside in the middle of the library, to divide the selected library into two different modules, Module A and Module B, with a difference in sequence at either the 5′ or 3′ end or both ends of the modules.
e) dividing the restricted library into two aliquots and specifically amplifying Module A from one aliquot and Module B from the second aliquot with sufficient PCR reactions to ensure maintenance of an average copy number of at least 100 copies of each sequence.
f) determining the frequency of each Module sequence within each selected library by dividing copy number by the total number of reads for that Module within each selected library.
g) multiplying the frequency of the sequences in Module A by the frequency of the sequences in Module B thus creating a matrix of the frequencies of 4 294 967 296 possible sequences for each selection,
h) performing steps b to g in at least duplicate to enable calculation of average frequencies and the standard deviation of these average frequencies for each 4 294 967 296 possible sequences for a given target and for selections with the same library in the absence of the target.
i) identifying sequences in terms of determining the statistical significance of the average differences in frequencies between a library selected for a target and a library selected in the absence of the target, including but not limited to evaluating Z values by subtracting the average frequency of each sequence in the absence of target from the average frequency of each sequence in the presence of target, and dividing this subtracted value by the average of the standard deviation for the same sequence in both the presence and absence of the target.

38. A method of treating a disorder or a disease in a subject in need thereof; comprising the steps of: i) diagnosing the disorder or disease using the aptamer or aptamers obtainable by the process according to claim 23; and ii) treating the subject being diagnosed during the step i).

39. A method of treating a disorder or a disease in a subject in need thereof; comprising the steps: i) diagnosing the disorder or disease using the aptamer according to claim 34; and ii) treating the subject being diagnosed during the step i).

40. A method for the detection and/or the quantification of target molecule comprising contacting a biological sample with the aptamer according to claim 34.


SEQUENCE LISTING (47 sequences)

SEQ ID NO: 1 - DNA, 27 bp, synthetic construct
  source 1..27: mol_type = other DNA
aaangaaannngaaacnnnaaacnttt

SEQ ID NO: 2 - DNA, 25 bp, synthetic construct
  source 1..25: mol_type = other DNA
taatacgactcactatagggataat

SEQ ID NO: 3 - DNA, 32 bp, synthetic construct
  source 1..32: mol_type = other DNA
tttcntttattatccctatagtgagtctatta

SEQ ID NO: 4 - DNA, 24 bp, synthetic construct
  source 1..24: mol_type = other DNA
gaacgagcgacctcatacgtattg

SEQ ID NO: 5 - DNA, 32 bp, synthetic construct
  source 1..32: mol_type = other DNA
caatacgtatgaggtcgctcgttcaaangttt

SEQ ID NO: 6 - DNA, 54 bp, synthetic construct
  source 1..54: mol_type = other DNA
aaangaaannngaatgnnnaaacntttaaangaaannncattcnnnttacntaa

SEQ ID NO: 7 - DNA, 27 bp, synthetic construct
  source 1..27: mol_type = other DNA
aaangaaannncattcnnnttacntaa

SEQ ID NO: 8 - DNA, 16 bp, synthetic construct
  source 1..16: mol_type = other DNA
tttcntttaaangttt

SEQ ID NO: 9 - DNA, 27 bp, synthetic construct
  source 1..27: mol_type = other DNA
aaangaaannngaatgnnnaaacnttt

SEQ ID NO: 10 - DNA, 33 bp, synthetic construct
  source 1..33: mol_type = other DNA
tttcntttattatccctatagtgagtcgtatta

SEQ ID NO: 11 - DNA, 25 bp, synthetic construct
  source 1..25: mol_type = other DNA
gaacgagcgacctcatacgtatttg

SEQ ID NO: 12 - DNA, 33 bp, synthetic construct
  source 1..33: mol_type = other DNA
caaatacgtatgaggtcgctcgttcaaangttt

SEQ ID NO: 13 - DNA, 33 bp, synthetic construct
  source 1..33: mol_type = other DNA
caaatacgtatgaggtcgctcgttcttangtaa

SEQ ID NO: 14 - DNA, 73 bp, synthetic construct
  source 1..73: mol_type = other DNA
ccagatacagacnngaggnnngaatnnnaaccatcggcgccaacannncattcnnncaganncagtagacagc

SEQ ID NO: 15 - DNA, 37 bp, synthetic construct
  source 1..37: mol_type = other DNA
caaatacgtatgaggtcgctcgttcccagatacagac

SEQ ID NO: 16 - DNA, 36 bp, synthetic construct
  source 1..36: mol_type = other DNA
taatacgactcactatagggataatgctgtctactg

SEQ ID NO: 17 - DNA, 61 bp, synthetic construct
  source 1..61: mol_type = other DNA
caaatacgtatgaggtcgctcgttcccagatacagacnngaggnnngaatnnnaaccatcg

SEQ ID NO: 18 - DNA, 62 bp, synthetic construct
  source 1..62: mol_type = other DNA
gcgccaacannncattcnnncaganncagtagacagcattatccctatagtgagtcgtatta

SEQ ID NO: 19 - skipped

SEQ ID NO: 20 - DNA, 39 bp, synthetic construct
  source 1..39: mol_type = other DNA
ggtcagacgtgtgctcttccgatcggggcgccgatggtt

SEQ ID NO: 21-22 - skipped (2 entries)

SEQ ID NO: 23 - DNA, 73 bp, synthetic construct
  source 1..73: mol_type = other DNA
ccagatacagaccggaggtctgaatctcaaccatcggcgccaacatttcattcaaccagaaccagtagacagc

SEQ ID NO: 24 - DNA, 73 bp, synthetic construct
  source 1..73: mol_type = other DNA
ccagatacagacacgaggtctgaatcctaaccatcggcgccaacagctcattcgcccagacccagtagacagc

SEQ ID NO: 25 - DNA, 73 bp, synthetic construct
  source 1..73: mol_type = other DNA
ccagatacagacctgaggtctgaatcttaaccatcggcgccaacaacgcattctgccagagtcagtagacagc

SEQ ID NO: 26 - DNA, 73 bp, synthetic construct
  source 1..73: mol_type = other DNA
ccagatacagaccggaggatcgaatctaaaccatcggcgccaacagcgcattcctccagacgcagtagacagc

SEQ ID NO: 27 - DNA, 73 bp, synthetic construct
  source 1..73: mol_type = other DNA
ccagatacagacctgaggtcggaatccaaaccatcggcgccaacagcgcattcctccagacgcagtagacagc

SEQ ID NO: 28 - DNA, 73 bp, synthetic construct
  source 1..73: mol_type = other DNA
ccagatacagactcgaggtctgaatctgaaccatcggcgccaacaccgcattcattcagacacagtagacagc

SEQ ID NO: 29 - DNA, 73 bp, synthetic construct
  source 1..73: mol_type = other DNA
ccagatacagaccggaggtctgaatccaaaccatcggcgccaacagcgcattcctccagacgcagtagacagc

SEQ ID NO: 30 - DNA, 73 bp, synthetic construct
  source 1..73: mol_type = other DNA
ccagatacagacccgaggcctgaatcccaaccatcggcgccaacagcgcattcctccagacgcagtagacagc

SEQ ID NO: 31 - DNA, 73 bp, synthetic construct
  source 1..73: mol_type = other DNA
ccagatacagaccagaggtctgaatcccaaccatcggcgccaacagggcattctggcagactcagtagacagc

SEQ ID NO: 32 - DNA, 73 bp, synthetic construct
  source 1..73: mol_type = other DNA
ccagatacagaccggaggtctgaatctcaaccatcggcgccaacagcgcattctagcagatacagtagacagc

SEQ ID NO: 33 - DNA, 73 bp, synthetic construct
  source 1..73: mol_type = other DNA
ccagatacagaccagaggtctgaatcgtaaccatcggcgccaacagcgcattcctccagacgcagtagacagc

SEQ ID NO: 34 - DNA, 27 bp, synthetic construct
  source 1..27: mol_type = other DNA
aaangwwwnnngwwwcnnnwwwcnttt

SEQ ID NO: 35 - DNA, 27 bp, synthetic construct
  source 1..27: mol_type = other DNA
aaangwwwnnngwwwgnnnwwwcnttt

SEQ ID NO: 36 - DNA, 27 bp, synthetic construct
  source 1..27: mol_type = other DNA
aaangwwwnnncwwwcnnnwwwcntaa

SEQ ID NO: 37 - DNA, 80 bp, synthetic construct
  source 1..80: mol_type = other DNA
aactcgcgagccacgggtagtcctccacctcactagggggttggcggatctcagctgacaaaacaagatctttgcgatcg

SEQ ID NO: 38 - DNA, 54 bp, synthetic construct
  source 1..54: mol_type = other DNA
ccctacacgacgctcttccgatctatcacgcaaatacgtatgaggtcgctcgtt

SEQ ID NO: 39 - DNA, 54 bp, synthetic construct
  source 1..54: mol_type = other DNA
aatgatacggcgaccaccgagatctacactctttccctacacgacgctcttccg

SEQ ID NO: 40 - DNA, 53 bp, synthetic construct
  source 1..53: mol_type = other DNA
caagcagaagacggcatacgagatgtgactggagttcagacgtgtgctcttcc

SEQ ID NO: 41 - DNA, 39 bp, synthetic construct
  source 1..39: mol_type = other DNA
ccctacacgacgctcttccgatctatcacggcgccaaca

SEQ ID NO: 42 - DNA, 63 bp, synthetic construct
  source 1..63: mol_type = other DNA
ggtcagacgtgtgctcttccgatcgggtaatacgactcactatagggataatgctgtctactg

SEQ ID NO: 43 - DNA, 73 bp, synthetic construct
  source 1..73: mol_type = other DNA
ccagatacagacttgaggtttgaatacgaaccatcggcgccaacattacattcccacagatccagtagacagc

SEQ ID NO: 44 - DNA, 36 bp, synthetic construct
  source 1..36: mol_type = other DNA
ccagatacagacttgaggtttgaatacgaaccatcg

SEQ ID NO: 45 - DNA, 37 bp, synthetic construct
  source 1..37: mol_type = other DNA
gcgccaacattacattcccacagatccagtagacagc

SEQ ID NO: 46 - DNA, 73 bp, synthetic construct
  source 1..73: mol_type = other DNA
ccagatacagaccagaggcgggaataagaaccatcggcgccaacaccacattcattcagatccagtagacagc

SEQ ID NO: 47 - DNA, 73 bp, synthetic construct
  source 1..73: mol_type = other DNA
ccagatacagacccgaggtgagaatttcaaccatcggcgccaacaaaacattctctcagatacagtagacagc